(19) United States
(12) Patent Application Publication
(10) Pub. No.: US 2023/0134195 A1
(43) Pub. Date: May 4, 2023

(54) SYSTEMS AND METHODS FOR VIDEO AND AUDIO ANALYSIS

(71) Applicant: AVAIL MEDSYSTEMS, INC., Santa Clara, CA (US)

(72) Inventors: Daniel HAWKINS, Palo Alto, CA (US); Ravi KALLURI, San Jose, CA (US); Arun KRISHNA, Pleasanton, CA (US); Shivakumar MAHADEVAPPA, Fremont, CA (US)

(21) Appl. No.: 18/046,684

(22) Filed: Oct. 14, 2022

Related U.S. Application Data
(63) Continuation of application No. PCT/US21/28180, filed on Apr. 20, 2021.
(60) Provisional application No. 63/012,390, filed on Apr. 20, 2020.

(51) Int. Cl. A61B 34/00; A61B 90/00

(52) U.S. Cl. CPC A61B 34/25; A61B 90/361; A61B 2017/00203

(57) ABSTRACT

Systems and methods for video analysis are provided. The systems and methods may utilize machine learning to recognize steps of a medical procedure as they are being performed, and compare them with expected steps. The systems and methods may aid in supporting a medical practitioner before the procedure, during the procedure, as well as providing feedback after the procedure has been completed.

### CROSS-REFERENCE

This application is a continuation of International Patent Application PCT/US21/28180, filed on Apr. 20, 2021, which claims priority to U.S. Provisional Application No. 63/012,390, filed on Apr. 20, 2020, each of which is incorporated herein by reference in its entirety for all purposes.

### BACKGROUND OF THE INVENTION

Medical practitioners may perform various procedures within a medical suite, such as an operating room. Oftentimes, there is little feedback or analysis regarding the steps taken by the medical practitioner and the timing. This can cause inefficiencies in the allocation of resources and does not provide many opportunities for medical practitioners to learn and improve.

Even if medical practitioners do wish to review the procedure and learn possible ways to increase efficiency and effectiveness, there are limited resources and options. Typical surgical videos may be lengthy and it may be difficult for practitioners to find the most relevant portions.

### SUMMARY OF THE INVENTION

A need exists for improved systems and methods of utilizing machine learning to analyze videos captured during medical procedures. A need exists for systems and methods that allow for medical practitioners to effectively and quickly review videos to watch relevant portions that may improve their medical technique. A further need exists for generating feedback to medical practitioners in real-time and after procedures have been completed.

Aspects of the invention are directed to a method of creating a highlight video of a medical procedure, said method comprising: recognizing one or more steps performed within a medical procedure based on video or audio data captured at the location of the medical procedure; assessing an amount of time to perform each of the one or more steps performed within the medical procedure; comparing the amount of time to perform each of the one or more steps in relation to a predicted amount of time to perform each of the one or more steps; and creating a highlight video of the medical procedure comprising video from the one or more steps where the amount of time to perform each of the one or more steps deviates from the predicted amount of time by more than a threshold.

In an aspect, the present disclosure provides a method of creating a highlight video of a medical procedure, said method comprising: recognizing one or more steps performed within a medical procedure based on video or audio data captured at the location of the medical procedure; assessing an amount of time to perform each of the one or more steps performed within the medical procedure; comparing the amount of time to perform each of the one or more steps in relation to a predicted amount of time to perform each of the one or more steps; and creating a highlight video of the medical procedure comprising video from the one or more steps where the amount of time to perform each of the one or more steps deviates from the predicted amount of time by more than a threshold. In some embodiments, the method may further comprise creating model video with audio before the medical procedure of the predicted medical procedure state with spatial and temporal analysis to assist medical personnel with performance of the medical procedure. In some embodiments, the method may further comprise creating automated documentation and transcripts using scene detection based on the video or audio data captured at the location of the medical procedure.

In some embodiments, the method may further comprise: predicting subsequent steps during the medical procedure to provide guidance for the medical procedure; indicating current and next steps during an active medical procedure; and modifying subsequent steps based on steps performed during the medical procedure, based on a success or accuracy rate.

In some embodiments, the highlight video is created or displayed during the medical procedure. In some embodiments, the highlight video is created or displayed after completion of the medical procedure. In some embodiments, the highlight video comprises an analysis of how one or more of the steps in the highlight video may be improved, when the amount of time to perform each of the one or more steps exceeds the predicted amount of time by more than a threshold. In some embodiments, the highlight video comprises an analysis of how one or more of the steps in the highlight video may be an area of strength for a practitioner performing the medical procedure, when the amount of time to perform each of the one or more steps falls below the predicted amount of time by more than a threshold.

In some embodiments, the video data is captured with aid of a medical console capable of communicating with a remote device, wherein the medical console comprises at least one camera supported by a movable arm. In some embodiments, the predicted amount of time to perform each of the one or more steps depends on an anatomy type of a subject of the medical procedure.

In some embodiments, the method may further comprise utilizing machine learning to recognize the one or more steps performed in the medical procedure and corresponding timing. In some embodiments, the method may further comprise utilizing machine learning to determine a predicted amount of time to perform each of the one or more steps. In some embodiments, selected data pertaining to a subject undergoing the medical procedure is automatically anonymized in the highlight video.

In another aspect, the present disclosure provides a system for creating a highlight video of a medical procedure, said system comprising one or more processors configured to, collectively or individually: recognize one or more steps performed within a medical procedure based on video or audio data captured at the location of the medical procedure; assess an amount of time to perform each of the one or more steps performed within the medical procedure; compare the amount of time to perform each of the one or more steps in relation to a predicted amount of time to perform each of the one or more steps; and create a highlight video of the medical procedure comprising video from the one or more steps where the amount of time to perform each of the one or more steps deviates from the predicted amount of time by more than a threshold.

In some embodiments, the system may further comprise one or more cameras or microphones configured to capture the video or audio data. In some embodiments, the one or more cameras are supported by a medical console configured to communicate with a remote device.

In another aspect, the present disclosure provides a method of identifying potential events during a medical procedure, said method comprising: recognizing one or more steps performed within a medical procedure based on video or audio data captured at the location of the medical procedure; assessing an amount of time to perform each of the one or more steps performed within the medical procedure; comparing the amount of time to perform each of the one or more steps in relation to a predicted amount of time to perform each of the one or more steps, wherein the predicted amount of time depends on an anatomy type of a subject of the medical procedure; and providing a notification of one or more steps where the amount of time to perform each of the one or more steps deviates from the predicted amount of time by more than a threshold. In some embodiments, the notification is provided to a medical practitioner performing the medical procedure in real-time. In some embodiments, the notification comprises guidance relating to the one or more steps deviating from the predicted amount of time by more than the threshold. In some embodiments, the notification is provided subsequent to completion of the medical procedure. In some embodiments, the notification comprises modified recommendations for a step when the amount of time to perform the step is less than the predicted amount of time by more than the threshold.

In another aspect, the present disclosure provides a method for video collaboration, the method comprising: (a) providing one or more videos of a surgical procedure to a plurality of users, wherein the one or more videos comprise at least one highlight video; and

(b) providing a virtual workspace for the plurality of users to collaborate based on the one or more videos, wherein the virtual workspace permits each of the plurality of users to (i) view the one or more videos or capture one or more recordings of the one or more videos, (ii) provide one or more telestrations to the one or videos or recordings, and (iii) distribute the one or more videos or recordings comprising the one or more telestrations to the plurality of users. In some embodiments, the virtual workspace permits the plurality of users to simultaneously stream the one or more videos and distribute the one or more videos or recordings comprising the one or more telestrations to the plurality of users. In some embodiments, the virtual workspace permits a first user to provide a first set of telestrations and a second user to provide a second set of telestrations simultaneously. In some embodiments, the virtual workspace permits a third user to simultaneously view the first set of telestrations and the second set of telestrations to compare or contrast inputs or guidance provided by the first user and the second user.

In some embodiments, the first set of telestrations and the second set of telestrations correspond to a same video, a same recording, or a same portion of a video or a recording. In some embodiments, the first set of telestrations and the second set of telestrations correspond to different videos, different recordings, or different portions of a same video or recording. In some embodiments, the at least one highlight video comprises a selection of one or more portions, stages, or steps of interest for the surgical procedure. In some embodiments, the first set of telestrations and the second set of telestrations are provided with respect to different videos or recordings captured by the first user and the second user. In some embodiments, the first set of telestrations and the second set of telestrations are provided or overlaid on top of each other with respect to a same video or recording captured by either the first user or the second user. In some embodiments, the virtual workspace permits each of the plurality of users to share one or more applications or windows at the same time with the plurality of users. In some embodiments, the virtual workspace permits the plurality of users to provide telestrations at the same time or modify the telestrations that are provided by one or more users at the same time. In some embodiments, the telestrations are provided on a live video stream of the surgical procedure or a recording of the surgical procedure.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a video capture system, in accordance with embodiments of the invention.

FIGS. 2A-2C show an example of an audio enhancement module, in accordance with embodiments of the invention.

FIG. 3 shows an example of a process to recognize steps in a medical procedure and detect timing disparities, in accordance with embodiments of the invention.

FIG. 4A shows an example of utilizing machine learning for step determination and recognition, in accordance with embodiments of the invention.

FIGS. 4B-4E show examples of various machine learning techniques that may be utilized, in accordance with embodiments of the invention.

FIG. 5 shows an example of generating different predicted steps based on recognized anatomy type, in accordance with embodiments of the invention.

FIG. 6 shows an example of a comparison between predicted steps for a procedure being compared with steps as they actually occur in real-time, in accordance with embodiments of the invention.

FIG. 7 shows an example of relevant portion of video being identified and brought up for viewing, in accordance with embodiments of the invention.

FIG. 8 shows an example of a user interface that may display images captured by one or more video cameras and provide support to a medical practitioner in real-time, in accordance with embodiments of the invention.

FIG. 9 shows an example of a user interface when personal data is removed, in accordance with embodiments of the invention.

FIG. 10 shows an example of factors that may be used to assess practitioner performance, in accordance with embodiments of the invention.

FIG. 11 shows an example of a system where multiple remote individuals may be capable of communicating with multiple health care facilities, in accordance with embodiments of the invention.

FIG. 12 shows an example of communication between different devices for a given entity, in accordance with embodiments of the invention.

FIG. 13 shows an exemplary computer system, in accordance with embodiments of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for video analysis. Various aspects of the invention described herein may be applied to any of the particular applications set forth below. The invention may be applied as a part of a health care system or communication system. It shall be understood that different aspects of the invention can be appreciated individually, collectively or in combination with each other.

A video system may aid in providing support to medical personnel who may be performing a medical procedure. The video system may be utilized to recognize anatomy type. A set of steps for a procedure for that anatomy type may be predicted, along with predicted timing for each step. The video system may capture images of a medical practitioner performing the procedure. The steps from the procedure may be recognized by the system. The timing of the steps as performed by the practitioner may be compared with the predicted timing for the steps. Significant disparities may be noted.

In some embodiments, the systems and methods provided herein may provide real-time support to the medical practitioner. While the medical practitioner is performing the procedure, helpful information for the procedure may be displayed and updated in real-time as steps are recognized. Any disparities from expected steps and/or timing may be noted to the medical practitioner.

The systems and methods provided herein may also provide post-procedure analysis and feedback. In some embodiments, a score for a practitioner's performance may be generated. The practitioner may be provided with an option to review the video, and the most relevant portions may be automatically recognized and brought to the front so that the practitioner does not need to spend extra time coming through irrelevant video.

The systems and methods provided herein may utilize a video capture system in order to capture images during the surgical procedure.

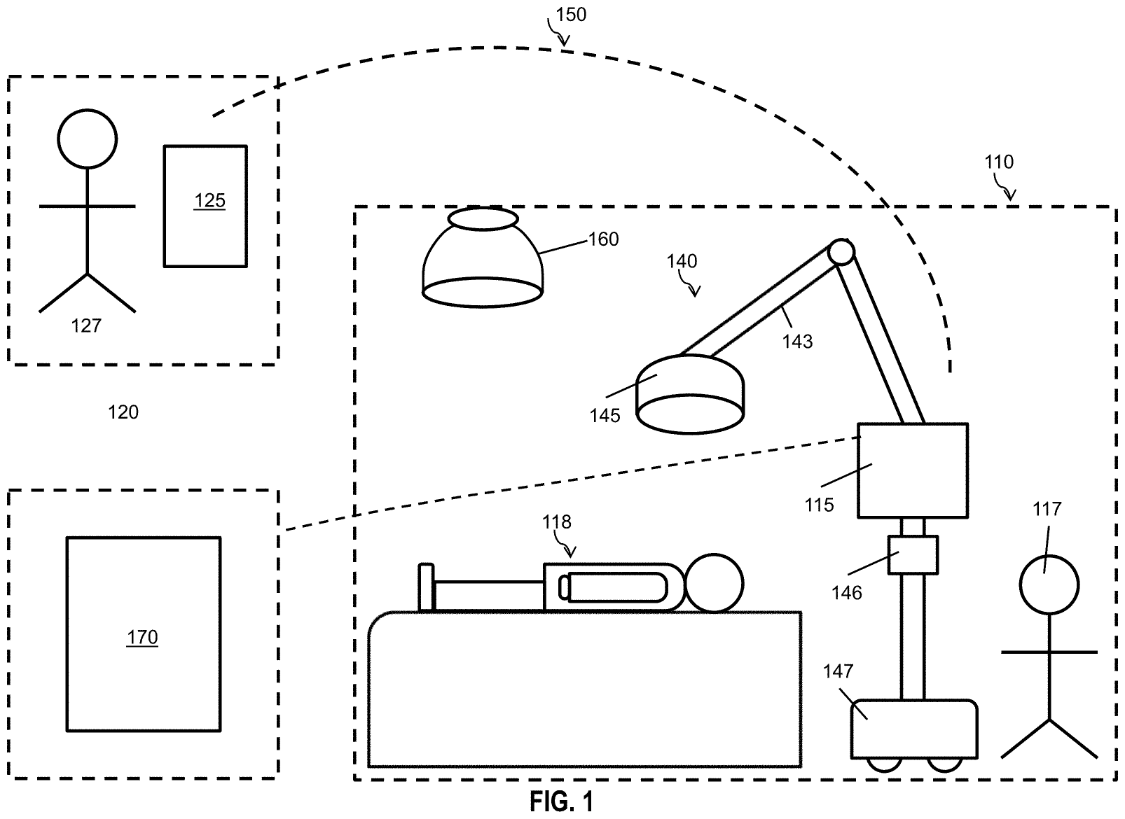

FIG. 1 shows an example of a video capture system utilized within a medical suite, such as an operating room. The video capture system may optionally allow for communications between the medical suite and one or more remote individuals, in accordance with embodiments of the invention. Communication may optionally be provided between a first location 110 and a second location 120.

The first location 110 may be a medical suite, such as an operating room of a health care facility. A medical suite may be within a clinic room or any other portion of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

The first location may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

Medical products may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

A video capture system may have one or more cameras. The video capture system may also comprise a local communication device 115. The local communication device may optionally communicate with a remote communication device 125.

One or more cameras may be integral to the communication device. Alternatively, the one or more cameras may be removable and/or connectable to the communication device. The one or more cameras may face a user when the user looks at a display of the communication device. The one or more cameras may face away from a user when the user looks at a display of the communication device. In some instances, multiple cameras may be provided which may face in different directions. The cameras may be capable of capturing images at a desired resolution. For instance, the cameras may be capable of capturing images at least a 6 mega pixel, 8 mega pixel, 10 mega pixel, 12 mega pixel, 20 mega pixel, 30 megapixels, 40 megapixels, or any number of pixels. The cameras may be capable of capturing SD, HD, Full HD, WUXGA, 2K, UHD, 4K, 8K, or any other level of resolution. A camera on a rep communication device may capture an image of a vendor representative. A camera on a local communication device may capture an image of a medical personnel. A camera on a local communication device may capture an image of a surgical site and/or medical tools, instruments or products.

The communication device may comprise one or more microphones or speakers. A microphone may capture audible sounds such as the voice of a user. For instance, the rep communication device microphone may capture the speech of the vendor representative and a local communication device microphone may capture the speech of a medical personnel. One or more speakers may be provided to play sound. For instance, a speaker on a rep communication device may allow a vendor representative to hear sounds captured by a local communication device, and vice versa.

In some embodiments, an audio enhancement module may be provided. The audio enhancement module may be supported by a video capture system. The audio enhancement module may comprise an array of microphones that may be configured to clearly capture voices within a noisy room while minimizing or reducing background noise. The audio enhancement module may be separable or may be integral to the video capture system.

A communication device may comprise a display screen. The display screen may be a touchscreen. The display screen may accept inputs by a user's touch, such as finger. The display screen may accept inputs by a stylus or other tool.

A communication device may be any type of device capable of communication. For instance, a communication device may be a smartphone, tablet, laptop, desktop, server, personal digital assistant, wearable (e.g., smartwatch, glasses, etc.), or any other type of device.

In some embodiments, a local communication device 115 may be supported by a medical console 140. The local communication device may be permanently attached to the medical console, or may be removable from the medical console. In some instances, the local communication device may remain functional while removed from the medical console. The medical console may optionally provide power to the local communication device when the local communication device is attached to (e.g., docked with) the medical console. The medical console may be mobile console that may move from location to location. For instance, the medical console may include wheels that may allow the medical console to be wheeled from location to location. The wheels may be locked into place at desired locations. The medical device may optionally comprise a lower rack and/or support base 147. The lower rack and/or support base may house one or more components, such as communication components, power components, auxiliary inputs, and/or processors.

The medical console may optionally include one or more cameras 145, 146. The cameras may be capable of capturing images of the patient 118, or portion of the patient (e.g., surgical site). The cameras may be capable of capturing images of the medical devices. The cameras may be capable of capturing images of the medical devices as they rest on a tray, or when they are handled by a medical personnel and/or used at the surgical site. The cameras may be capable of capturing images at any resolution, such as those described elsewhere herein. The cameras may be used to capture a still images and/or video images. The cameras may be capturing images in real time.

One or more of the cameras may be movable relative to the medical console. For instance, one or more cameras may be supported by an arm. The arm may include one or more sections. In one example, a camera may be supported at or near an end of an arm. The arm may include one or more sections, two or more section, three or more sections, four or more sections, or more sections. The sections may move relative to one another or a body of the medical console. The sections may pivot about one or more hinge. In some embodiments, the movements may be limited to a single plane, such as a horizontal plane. Alternatively, the movements need not be limited to a single plane. The sections may move horizontally and/or vertically. A camera may have at least one, two, three, or more degrees of freedom. An arm may optionally include a handle that may allow a user to manually manipulate the arm to a desired position. The arm may remain in a position to which it has been manipulated. A user may or may not need to lock an arm to maintain its position. This may provide a steady support for a camera. The arm may be unlocked and/or re-manipulated to new positions as needed. In some embodiments, a remote user may be able to control the position of the arm and/or cameras.

In some cases, the cameras and/or imaging sensors of the present disclosure may be provided separately from and independent of the medical console or one or more displays. The cameras and/or imaging sensors may be used to capture images and/or videos of an ongoing surgical procedure or a surgical site that is being operated on, that has been operated on, or that will be operated on as part of a surgical procedure. In some cases, the cameras and/or imaging sensors disclosed herein may be used to capture images and/or videos of a surgeon, a doctor, or a medical worker assisting with or performing one or more steps of the surgical procedure. The cameras and/or imaging sensors may be moved independently of the medical console or one or more displays. For instance, the cameras and/or imaging sensors may be positioned and/or oriented in a first direction or towards a first region, and the medical console or the one or more displays may be positioned and/or oriented in a second direction or towards a second region. In some cases, the one or more displays may be moved independently of the one or more cameras and/or imaging sensors without affecting or changing a position and/or orientation of the cameras or imaging sensors. The one or more displays described herein may be used to display the images and/or videos captured using the cameras and/or imaging sensors. In some cases, the one or more displays may be used to display images, videos, or other information or data provided by a remote vendor representative to one or more medical workers in a healthcare facility or an operating room where a surgical procedure may be performed or conducted. The images or videos displayed on the one or more displays may comprise an image or a video of a vendor representative. The images or videos displayed on the one or more displays may comprise images and/or videos of the vendor representative as the vendor representative provides live feedback, instructions, guidance, counseling, or demonstrations. Such live feedback, instructions, guidance, counseling, or demonstrations may relate to a usage of one or more medical instruments or tools, or a performance of one or more steps in a surgical procedure using the one or more medical instruments or tools.

In some embodiments, the one or more cameras and/or imaging sensors may comprise two or more cameras and/or imaging sensors. The two or more cameras and/or imaging sensors may be moved independently of each other. In some cases, a first camera and/or imaging sensor may be movable independently of and relative to a second camera and/or imaging sensor. In some cases, the second camera and/or imaging sensor may be fixed or stationary. In other cases, the second camera and/or imaging sensor may be movable independently of and relative to the first camera and/or imaging sensor.

In some embodiments, one or more cameras may be provided at the second location. The one or more cameras may or may not be supported by the medical console. In some embodiments, one or more cameras may be supported by a ceiling 160, wall, furniture, or other items at the second location. For instance, one or more cameras may be mounted on a wall, ceiling, or other device. Such cameras may be directly mounted to a surface, or may be mounted on a boom or arm. For instance, an arm may extend down from a ceiling while supporting a camera. In another example, an arm may be attached to a patient's bed or surface while supporting a camera. In some instances, a camera may be worn by medical personnel. For instance, a camera may be worn on a headband, wrist-band, torso, or any other portion of the medical personnel. A camera may be part of a medical device or may be supported by a medical device (e.g., endoscope, etc.). The one or more cameras may be fixed cameras or movable cameras. The one or more cameras may be capable of rotating about one or more, two or more, or three or more axes. The one or more cameras may include pan-tilt-zoom cameras. The cameras may be manually moved by an individual at the location. The cameras may be locked into position and/or unlocked to be moved. In some instances, the one or more cameras may be remotely controlled by one or more remote users. The cameras may zoom in and/or out. Any of the cameras may have any of the resolution values as provided herein. The cameras may optionally have a light source that may illuminate an area of interest. Alternatively, the cameras may rely on external light sources.

Images captured by the one or more cameras 145, 146 may be analyzed as described further elsewhere herein. The video may able analyzed in real-time. The videos may be sent to a remote communication device. This may allow a remote use to remotely view images captured by the field of view of the camera. For instance, the remote user may view the surgical site and/or any medical devices being used. The remote user may be able to view the medical personnel. The remote user may be able to view these in substantially real-time. For instance, this may be within 1 minutes or less, 30 seconds or less, 20 seconds or less, 15 seconds or less, 10 seconds or less, 5 seconds or less, 3 seconds or less, 2 seconds or less, or 1 second or less of an event actually occurring.

This may allow a remote user to lend aid or support without needing to be physically at the first location. The medical console and cameras may aid in providing the remote user with the necessary images and information to have a virtual presence at the first location.

The video analysis may occur locally at the first location 110. In some embodiments, the analysis may occur on-board a medical console 140. For instance, the analysis may occur with aid of one or more processors of a communication device 115 or other computer that may be located at the medical console. In some instances, the video analysis may occur remotely from the first location. In some instances, one or more servers 170 may be utilized to perform video analysis. The server may be able to access and/or receive information from multiple locations and may collect large datasets. The large datasets may be used in conjunction with machine learning in order to provide increasingly accurate video analysis. Any description herein of a server may also apply to any type of cloud computing infrastructure. The analysis may occur remotely and feedback may be communicated back to the console and/or location communication device in substantially real-time. Any description herein of real-time may include any action that may occur within a short span of time (e.g., within less than or equal to about 10 minutes, 5 minutes, 3 minutes, 2 minutes, 1 minute, 30 seconds, 20 seconds, 15 seconds, 10 seconds, 5 seconds, 3 seconds, 2 seconds, 1 second, 0.5 seconds, 0.1 seconds, 0.05 seconds, 0.01 seconds, or less).

In some embodiments, medical personnel may communicate with one or more remote individuals.

A second location 120 may be any location where a remote individual 127 is located. The second location may be remote to the first location. For instance, if the first location is a hospital, the second location may be outside the hospital. In some instances, the first and second locations may be within the same building but in different rooms, floors, or wings. The second location may be at an office of the remote individual. A second location may be at a residence of a remote individual.

A remote individual may have a remote communication device 125 which may communicate with a local communication device 115 at the first location. Any form of communication channel 150 may be formed between the rep communication device and the location communication device. The communication channel may be a direct communication channel or indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

Communications between rep communication devices and local communication devices may be encrypted. Optionally, only authorized and authenticated rep communication devices and local communication devices may be able to communicate over a communication system.

In some embodiments, a remote communication device and/or local communication device may communicate with one another through a communication system. The communication system may facilitate the connection between the remote communication device and the local communication device. The communication system may aid in accessing scheduling information at a health care facility. The communication system may aid in presenting, on a remote communication device, a user interface to a remote individual about one or more possible medical procedures that may benefit from the remote individual's support.

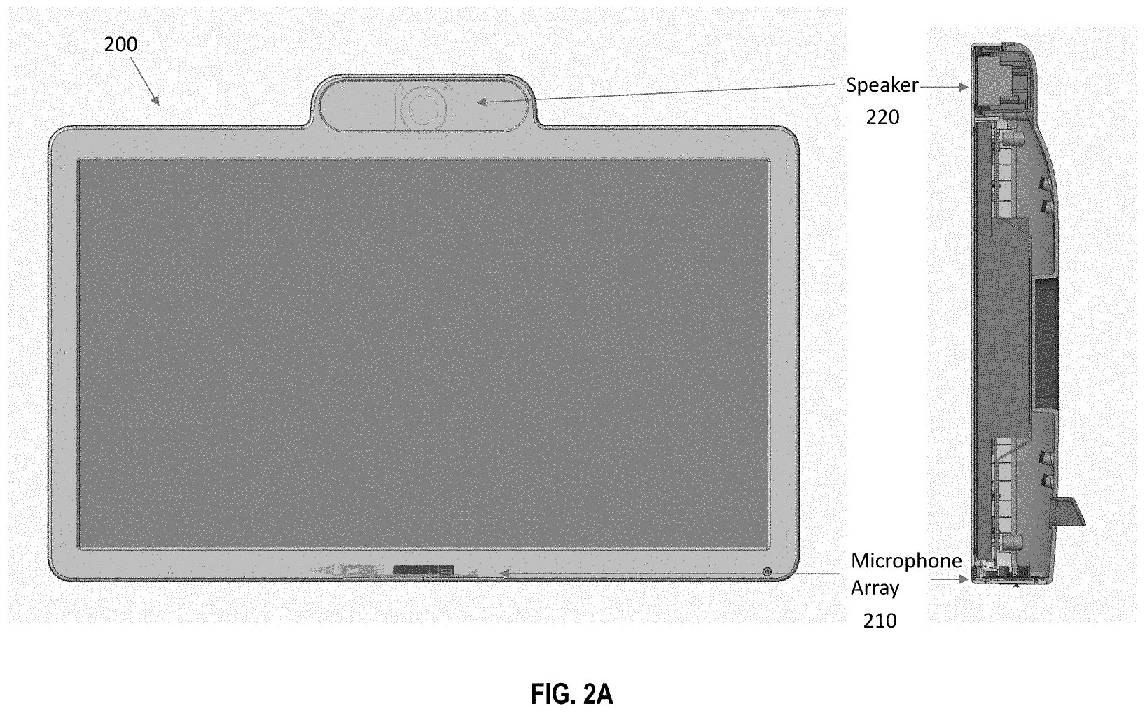

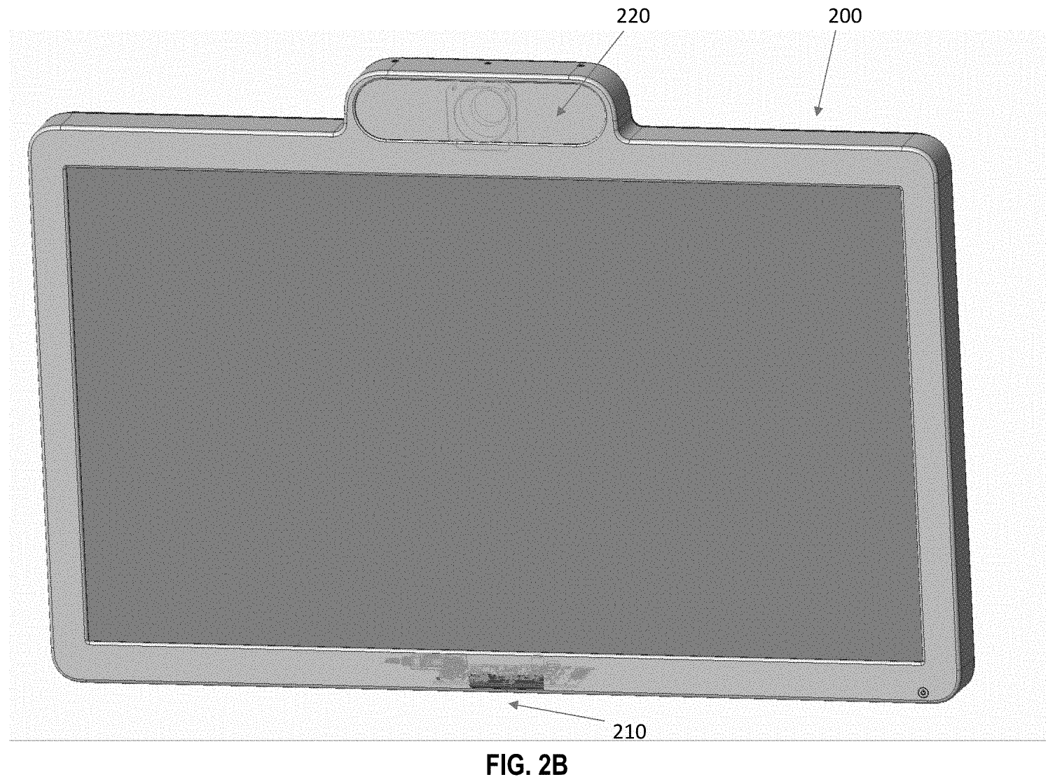

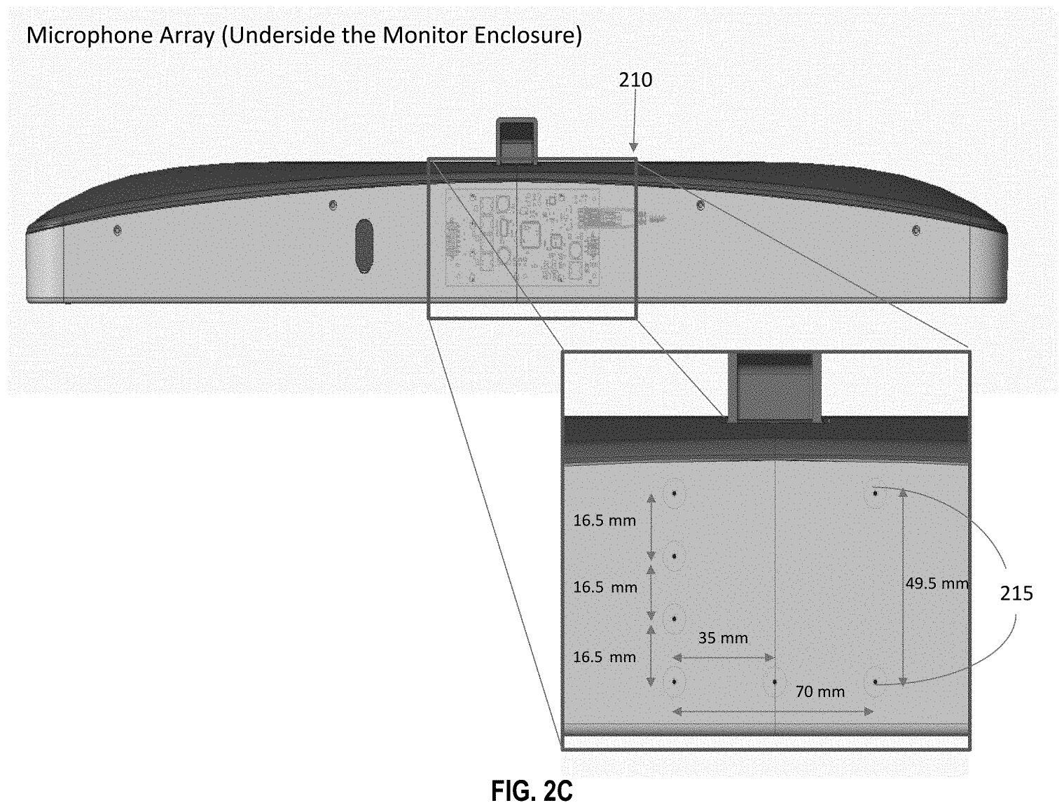

FIGS. 2A-2C show an example of an audio enhancement module, in accordance with embodiments of the invention. One or more microphones may be utilized at a location to collect audio. The audio enhancement module may comprise a plurality of microphones. The plurality of microphones may be referred to as a microphone array. Any description herein of a microphone area may refer to any type of audio enhancement module and vice versa.

FIG. 2A shows an example of a microphone array 210 and speaker 220 that may be incorporated into a display 200. For example, a speaker and microphone array may be placed on-board a screen or a communication device as described herein. The screen and/or communication device may optionally be provided on a medical console that may have one or more wheels to roll it around. Any description herein of a display may apply to a screen or any type of communication device.

In some embodiments, a speaker and microphone array may be placed at opposing ends of the display. For example, the speaker may be positioned at or near a top of the display and the microphone array may be placed at or near a bottom of the display, or vice versa. Similarly, a speaker may be placed at or near a right side of the display and the microphone array may be placed at or near a left side of the display, or vice versa. Positioning the speaker and microphone array at opposing ends may advantageously allow the noise coming from the speaker to not or minimally interfere with the sound captured by the microphone array.

The speaker may be positioned so that sound is directed forwards, away from a direction of the display. The sound may be directed toward an individual facing a screen. This may allow an individual to engage in communications with a remote individual.

The microphone array may optionally be positioned to collect voice audio. In some embodiments, the microphone array may be configured to minimize or reduce background noise.

FIG. 2B shows a perspective view of a display 200 with a speaker 220 and a microphone array 210.

FIG. 2C shows a close-up of a microphone array 210. In some embodiments, the microphone array may be positioned on a bottom surface of the display. The microphone array may be positioned at or near (e.g., within 10%, 5%, 3% or 1% of the bottom surface of the display). The microphone array may be positioned at or near any other side of the display.

The microphone array 210 may comprise a plurality of microphones 215. The microphones may be arranged in one or more rows, one or more columns, and/or one or more arrays. The microphones may or may not have a staggered arrangement or a randomized arrangement. Any description herein of a microphone array may refer to any arrangement of microphones. Any number of microphones may be provided. For instance, one or more, two or more, three or more, four or more, five or more, six or more, seven or more, eight or more, nine or more, ten or more, twelve or more, fifteen or more, twenty or more, thirty or more, or fifty or more microphones may be utilized in a microphone array. In some embodiments, the number of microphones may be less than any of the numbers provided herein or fall within a range between any two of the numbers provided herein.

The microphones may be provided along with a full range speaker. For example, seven microphones and a full range speaker may be provided as illustrated. The transmit path may accept inputs from the microphones (e.g., seven microphones) (sᵢₙ) and the loudspeaker references signal (refᵢₙ). The transmit path processing may cancel or reduce acoustic echo on the microphone inputs. The transmit path processing may perform beamforming for direction-finding and/or spatial filtering. The transmit path processing may apply to spectral noise reduction. The transmit path processing may apply gain to the final output.

A receive path may apply gain and equalization to an input signal (rᵢₙ) prior to output by the speaker (rₒᵤₜ). Under certain circumstances, the receive path may also modulate the gain to improve full-duplex performance.

Spatial filter beams and direction-finding beams may be provided. The direction-finding beams may comprise delay and sum beams, and there may be twelve (or any other number) evenly spaced directions. For example, when twelve directions are provided, the beams may be spaced out like a clock dial.

The spatial filter beams may comprise at least six beams, eight beams, ten beams, twelve beams, or any other number of beams. For instance, the beams may comprise (a) a narrow forward-facing beam, (2) a narrow beam pointing 30 degrees to the right of the medical console—or 1 O'clock, (3) a narrow beam pointing 30 degrees to the left of the medical console—or 11 O'clock, (4) a broader forward-facing beam that has good pickup +/−60 degrees, (5), an omni-directional beam using only microphone #0, (6) a fallback beam (similar or same as #4 beam) and/or a (7) a rear-facing beam to serve as the noise reference. The beams may be configurable and any combination of beams may be utilized. Any other number of spatial filter beams may be provided. They may have similar arrangements or ranges as described herein. The degree values may vary by less than or equal to 1%, 5%, 10%, 20%, 30%, 50%, or 70% of the values described herein.

In an automatic search mode, the most confident direction may be identified. The mapping of the direction to beams works as follows:
1. direction #0 maps to forward narrow beam
2. direction #1 maps to forward beam 30 degrees to right
3. direction #11 maps to forward beam 30 degrees to left
4. directions 2 through 10 map to broader beam facing forward

If the search figure of merit (aka confidence) is low, then the fallback beam is selected. In some embodiments, there is no automatic way to select the omni beam.

The dimensions are provided by way of example only and are not limiting. In some embodiments, the dimensions may vary by less than or equal to 1%, 3%, 5%, 10%, 15%, 20%, 25%, 30%, or 40%. In some embodiments, the proportion of the distance between the microphones may remain fixed or within 1%, 3%, 5%, 10%, 15%, 20%, 25%, 30%, or 40%, even if distances vary.

Any description herein of a video analysis may also apply to audio analysis as well. The audio analysis may occur separately or may occur in conjunction with the video analysis. The timing of the audio may be known relative to the timing of one or more videos. The audio may be analyzed within the context of the video collected at the same time, and vice versa.

In some embodiments, the captured audio may be converted and/or analyzed. For instance, speech may be converted to text. The text may be used for a transcript of the activities that transpired. The speech recognition may be able to identify the speaker based on the speaker's voice. The transcript may include information about the identity of the individual speaking for each item of text. Timing data may be associated with the text so that the timing of the speech can be analyzed. Similarly, other noises may be analyzed and/or recognized. For example, the sound of a falling object may be detected and noted. In another example particular instruments may make different noises, and the use of such instruments may be detected and/or analyzed based on the audio.

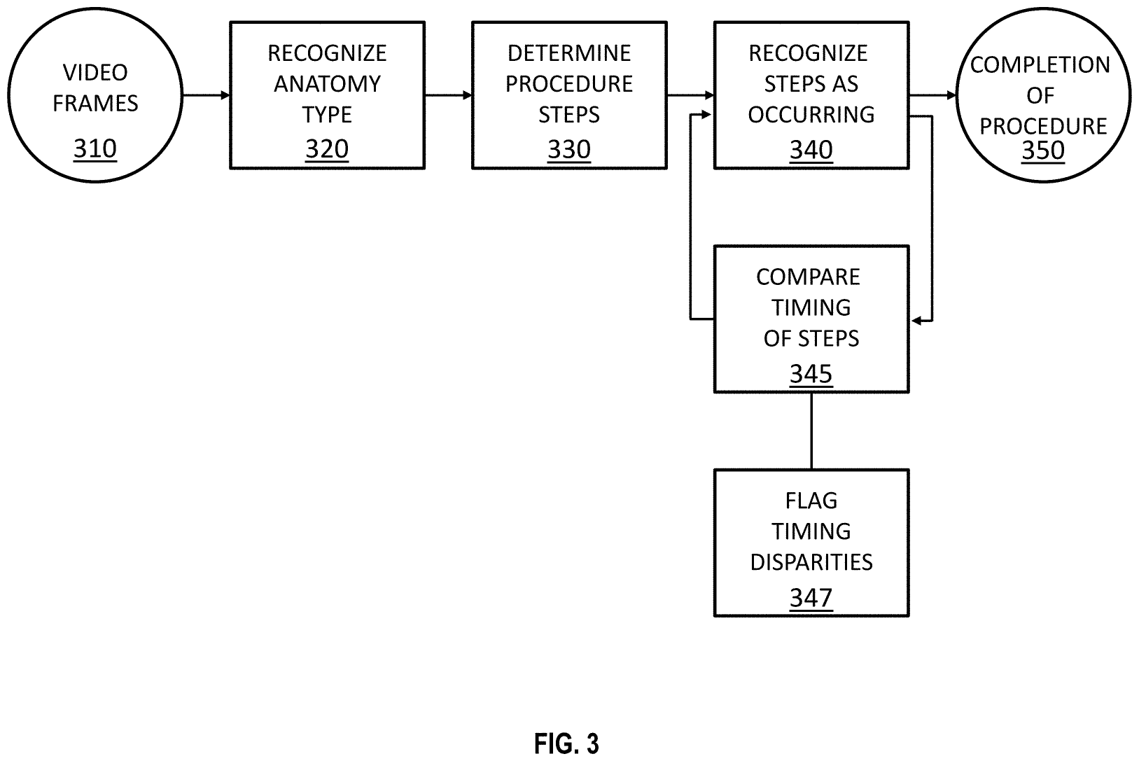

FIG. 3 shows an example of a process to recognize steps in a medical procedure and detect timing disparities, in accordance with embodiments of the invention. Video frames may be obtained and analyzed 310. Anatomical type of a patient may be recognized 320. Based on the anatomy type, one or more predicted procedure steps may be determined 330. As medical personnel perform the procedure, the steps may be recognized in real-time 340. While the steps are being performed, the timing of the steps may be recognized and compared with predicted timing of the steps 345. Any disparities in actual timing of the steps as compared to predicted timing may be flagged 347. The surgery may be completed after the steps are performed 350.

Video frames may be obtained 310. Video may be captured during a procedure. The video may be captured with aid of one or more cameras present at a location of the procedure. The one or more cameras may include cameras supported by a medical console, and/or one or more cameras that are not supported by the medical console (e.g., on a wall, ceiling, furniture, wearable, etc.). In some embodiments, video from a single camera may be analyzed. Alternatively, video captured by multiple cameras may be analyzed together. In addition, external imaging inputs such as ultrasound, endoscopy. elastography, ECG, fluoroscopy, medical photography, tactile imaging, thermography, da Vinci Surgical System, medical imaging inputs, and so forth, can be connected and video frames of these external imaging may also be analyzed. Timing information between the various video cameras may be synchronized in order to get a sense of comparative timing between the images captured by each of the video cameras. For instance, each camera may have an associated clock or may communicate with a clock. Such clocks may be synchronized with one another in order to accurately determine the timing of the images captured by multiple video sources. In some instances, multiple video cameras may communicate with a single clock. In some instances, the timing on the clocks may be different, but a disparity between the clocks can be known. The disparity between the clocks can be used to ensure that the video being analyzed from multiple video cameras are using the proper timing.

An anatomy type of a patient may be recognized 320. In some embodiments, images from the video may be used to recognize anatomical type of the patient. In some instances, a patient's medical records may be automatically accessed and used to aid in recognition of the anatomical type of the patient. In some instances, medical personnel may input information that may be used to determine a patient's anatomy type. In some instances, the medical personnel may directly input the patient's anatomy type. In some instances, information from multiple sources (e.g., two or more of video images, medical records, manual input) may be used to determine the patient's anatomy type. Examples of factors that may affect a patient's anatomy type may include, but is not limited to, gender, age, weight, height, positioning of various anatomical features, size of various anatomical features, past medical procedures or history, presence or absence of scar tissue, or any other factors.

Video may be analyzed and used to aid in determining a patient's anatomy type. Object recognition may be utilized to recognize different anatomical features on a patient. In some instances, one or more feature points may be recognized and used to recognize one or more objects. In some embodiments, size and/or scaling may be determined between the different anatomical features. One or more fiducial markers may be provided on a patient to aid in determining scale and/or size.

In some embodiments, machine learning may be utilized in determining a patient's anatomy type. The systems and methods provided herein may utilize training datasets in determining anatomy type. When the patient's information is provided and/or accessed, the systems and methods provided herein may automatically determine the patient's anatomy type. In some embodiments, the determined anatomy type may optionally be displayed to medical personnel. The medical personnel may be able to review the determined anatomy type and confirm whether the assessment is accurate. If the assessment is not accurate, the medical personnel may be able to correct the anatomy type or provide additional information that may update the anatomy type.

Determining the anatomical type of a patient may be useful in determining procedure steps 330. For instance, patients with different anatomical types may require different steps in order to achieve a similar goal. Medical personnel may take different steps depending on a patient's placement or size of various anatomical features, age, past medical conditions, overall health, or other factors. In some instances, different steps may be taken for different anatomical types. For instances, certain steps or techniques may be better suited for particular anatomical features. In other instances, the same steps may be taken, but the timing may differ significantly. For instance, for particular anatomical features, a particular step may be more difficult to perform, and may end up typically taking a longer time than if the anatomical feature was different.

In some embodiments, machine learning may be utilized in determining the steps to utilize for a particular anatomy type. The systems and methods provided herein may utilize training datasets in determining determine the steps that are typically used for a particular anatomy type. This may include determining timing of the various steps that are used. In some instances, the recommended steps may be displayed to the medical personnel. The steps may be displayed to the medical personnel before the medical personnel starts the procedure. The medical personnel may be able to review the recommended steps to confirm whether these recommendations are accurate. If the recommendation is not accurate or desirable, the medical personnel may provide some feedback or change the steps. The display may or may not include information about expected timing for the various steps.

In combination with machine learning technologies & data analytics, optimum techniques for performing a surgery (or any other medical procedure) may be determined. Post-surgery recovery time may be measured, and determinations may be made what steps the medical personnel took, and what tools (e.g., medical products) were used such that a patient can have less recovery time. The systems and methods provided herein may provide recommendation to the medical personnel, such as doctors, specialist and representatives live, on demand and offline modes.

The video analysis system may recognize steps as a medical personnel performs the procedure. The video may be analyzed to recognize the tools used by the medical personnel and/or actions taken by the medical personnel. Object recognition may be utilized to recognize the tools used and/or steps taken by the medical personnel. For example, if the first step is to make an incision, the video may be analyzed to recognize the scalpel used to make the incision and/or recognize the site at which the incision is made. In some instances, the motion of a medical personnel and/or medical personnel's hands may be used to recognize that an incision is being made.

The video may be analyzed to recognize the various steps taken by the medical personnel and compare them with the predicted steps 345. The video analysis may include be used to determine if the medical personnel conducts a different step from the predicted step. For example, if the medical personnel is expected to open a vessel, but the medical personnel instead performs a different step, such a difference may be flagged 347. In some instances, a visual or audio indicator may be provided to the medical personnel as soon as the disparity is detected. For example, a message may be displayed on the screen indicating that the medical personnel is deviating from the plan. The message may include an indication of the predicted step and/or the actual detected step occurring. Optionally, an audio message may provide similar information. For example, an audio message may indicate a deviation has been detected from the predicted step. An indication of the details of the predicted step and/or detected deviation may or may not be provided. Such feedback may be provided in real-time while the medical personnel is performing the procedure. This may advantageously allow the medical personnel to assess and make any corrections or adjustments if necessary.

The comparison may include comparison of timing. For example, even if the steps that are performed match up, the video may be analyzed to detect if there is significant deviation from expected timing of the step. For example, it may be expected that step 1 typically takes about 5 minutes to perform. If the step ends up taking 15 minutes, this difference in timing may be recognized 345 and/or flagged 347. When a significant difference in time is provided, a message (e.g., visual and/or audio message) may optionally be provided to the medical personnel. For instance, if a step is taking longer than expected, a display may show information that may aid the medical personnel in performing the step. Helpful hints or suggestions may be provided in real-time. In some embodiments, the timing information may be tracked in order to update a prediction of a timing of the surgery. In some instances, updates to expected timing and/or the percentage of completion of a procedure may be provided to a medical personnel while the medical personnel is performing the procedure.

In some embodiments, the degree of discrepancy for timing before flagging the discrepancy may be adjustable. For instance, if an average step takes about 15 minutes, but the medical personnel takes 16 minutes to perform the step, the degree of discrepancy may not be sufficient enough to make a note or raise a flag. In some instances, the degree of discrepancy needed to raise a flag may be predetermined. In some instances, the degree of discrepancy to reach a threshold to raise a flag may be on an absolute time scale (e.g., number of minutes, number of seconds). In some instances, the degree of discrepancy to reach a threshold to raise a flag may be on a relative time scale (e.g., percentage of amount of time that a step typically takes). The threshold value may be fixed, or may be adjustable. In some embodiments, a medical personnel may provide a preferred threshold (e.g., if the discrepancy exceeds more than 5 minutes, or more than 20% of expected procedure time). In other embodiments, the threshold may be set by an administrator of a health care facility, or another group member that supervises or works with the medical personnel.

The procedure may be completed when the medical personnel has completed all of the steps 350. In some embodiments, the medical may provide an input to indicate that the procedure has been completed. When the surgery has been completed, the video may be analyzed and/or a shortened or condensed version of the video may be completed. The medical personnel may be able to view the video in its raw form or in its condensed form after completion of the procedure. In some embodiments, the video analysis system may automatically provide feedback to the medical personnel regarding the execution of the procedure. For instance, the analysis system may automatically indicate if significant deviations in steps and/or timing occurred. In some instances, the system may provide recommendations to the medical personnel on differences that can be made by the medical personnel to improve efficiency and/or effectiveness of the procedure. Optionally, a score or assessment may be provided for the medical personnel's completion of the procedure.

Any description herein of the video analysis may also include audio analysis. As previously described, audio systems may be used to capture sound during a procedure. Optionally, an audio enhancement module may be used to clearly capture sounds from the voice of a medical personnel. The words spoken by the medical personnel may be analyzed and/or recognized. For instance, the medical personnel may announce various steps that are occurring while the medical personnel performs the step. The audio information may be analyzed in conjunction with the video information to aid in anatomy recognition, determining predicted steps, and/or recognizing current steps.

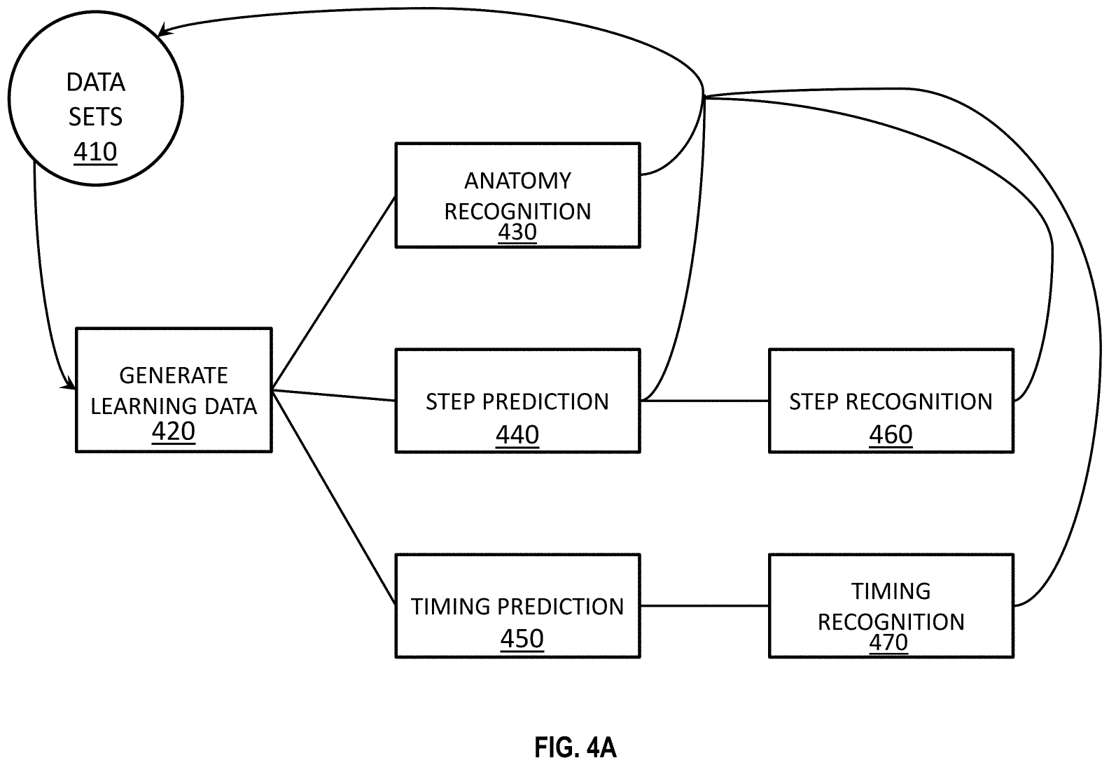

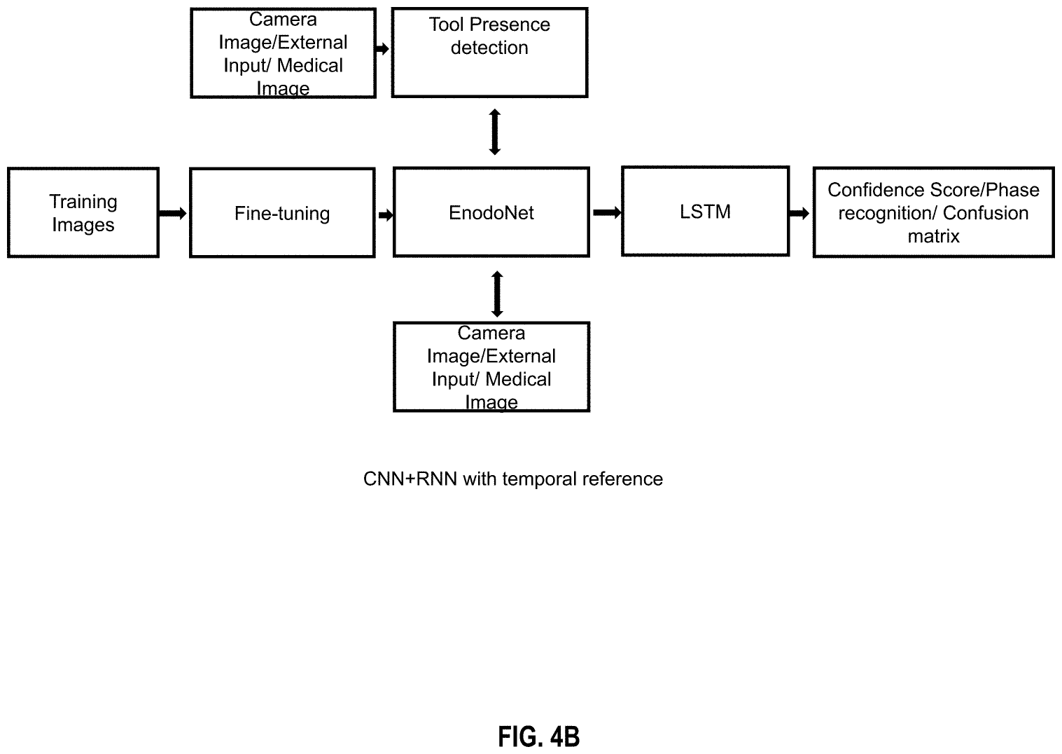

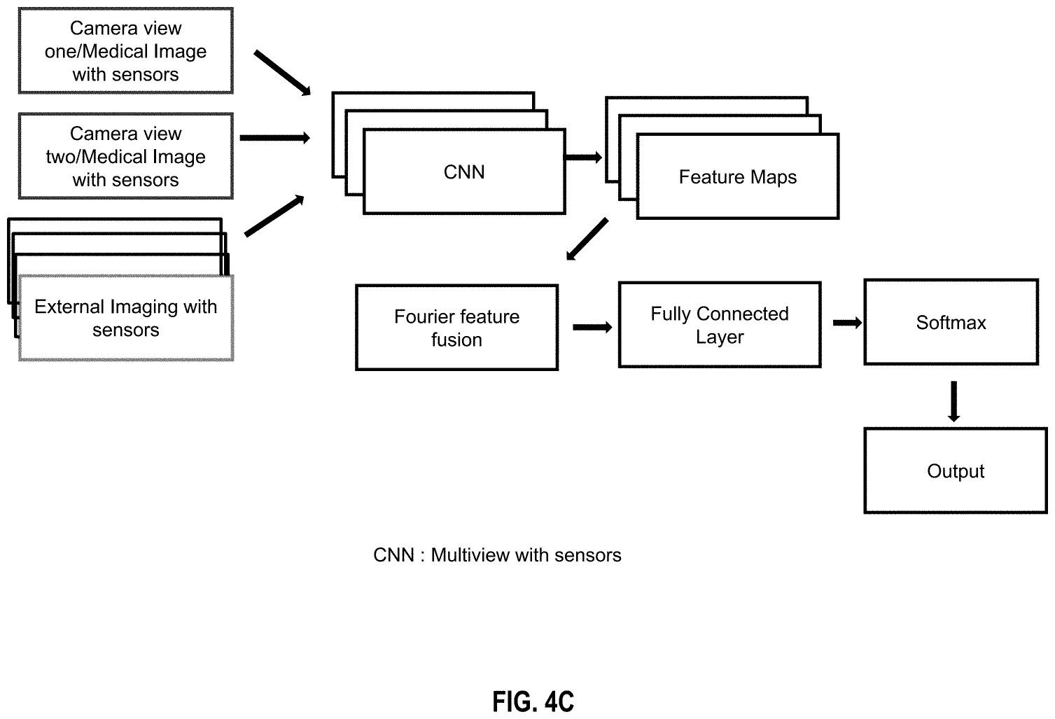

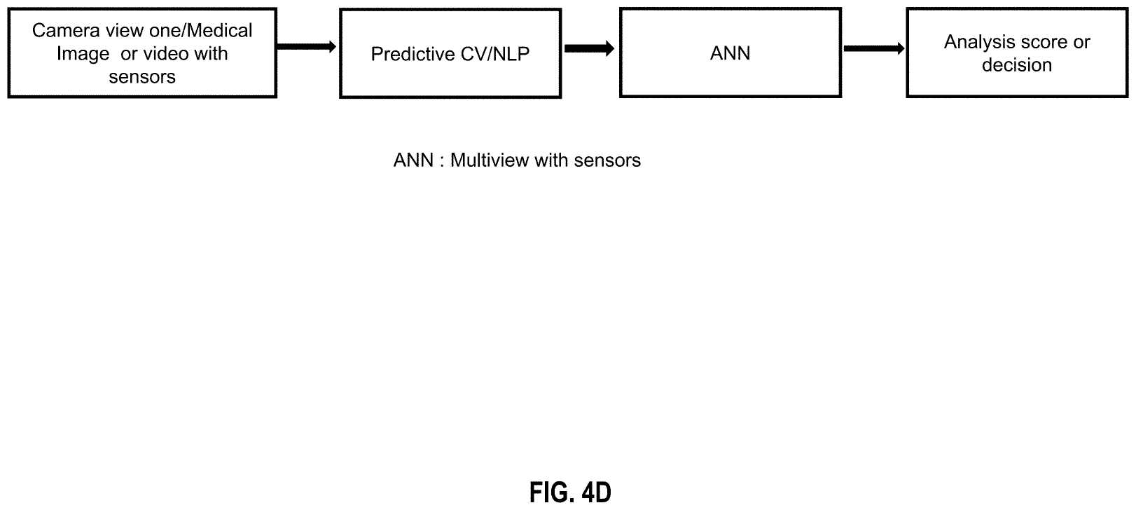

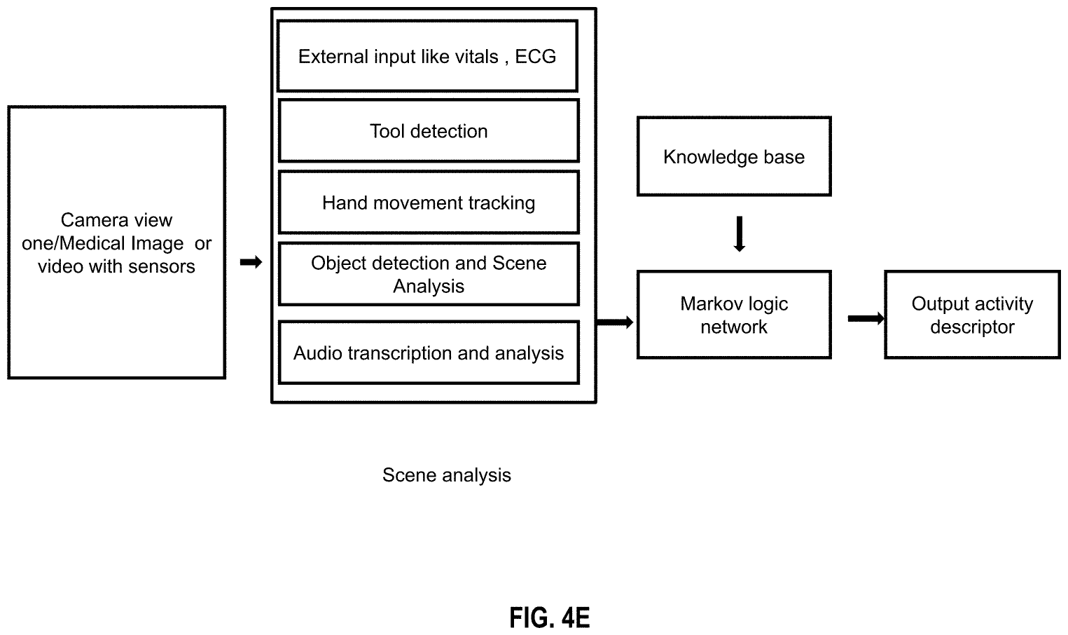

FIG. 4A shows an example of utilizing machine learning for step determination and recognition, in accordance with embodiments of the invention. Any description herein of machine learning may apply to artificial intelligence, and vice versa, or any combination thereof. One or more data sets may be provided 410. Machine learning data may be generated based on the data sets 420. The learning data may be useful for anatomy recognition 430, step prediction 440, and timing prediction 450. Machine learning may be useful for step recognition 460 and timing recognition 470 as well. The data from anatomy recognition, step prediction, step recognition, timing prediction and/or timing recognition may be fed back into the data sets to improve the machine learning algorithms.

One or more data sets may be provided 410. In some embodiments, data sets may advantageously include a large number of examples collected from multiple sources. In some embodiments, the video analysis system may be in communication with multiple health care facilities and may collect data over time regarding procedures. The data sets may include anatomical data about the patients, procedures performed and associated timing information with the various steps of the procedures. As medical personnel perform additional procedures, data relating to these procedures (e.g., anatomy information, procedure/step information, and/or timing information) may be constantly updated and added to the data sets. This may improve the machine learning algorithm and subsequent predictions over time.

The one or more data sets may be used as training data sets for the machine learning algorithms. Learning data may be generated based on the data sets. In some embodiments, supervised learning algorithms may be used. Optionally, unsupervised learning techniques and/or semi-supervised learning techniques may be utilized in order to generate learning data 420.

In some embodiments, the machine learning may be used to improve anatomy recognition 430. In some embodiments, video captured from one or more cameras during the medical procedure may be analyzed to detect an anatomy type for a patient. Optionally, audio data, medical records, or inputs by medical personnel may be used in addition or alternatively in order to determine an anatomy type for a patient. In some embodiments, object recognition and/or sizing/scaling techniques may be used to determine an anatomy type for a patient. A medical personnel may or may not provide feedback in real-time whether the anatomy type that was predicted using the video analysis was correct. In some embodiments, the feedback may be useful for improving anatomy recognition in the future.

In some embodiments, the various steps for a medical procedure may be predicted 440 using a machine learning algorithm. In some embodiments, video information, audio data, medical records, and/or inputs by medical personnel may be used alone or in combination to predict the steps for the medical procedure to be performed by the medical personnel. In some embodiments, the steps may vary depending on an anatomy type of the patient. Machine learning may be useful for generating a series of predicted steps for the procedure based on the collected information. Optionally, medical personnel may or may not provide feedback in real-time whether the predicted steps are correct for the particular patient. In some embodiments, the feedback may be useful for improving step prediction in the future.

In some embodiments, the timing of the various steps for a medical procedure may be predicted 450 using a machine learning algorithm. In some embodiments, video information, audio data, medical records, and/or inputs by medical personnel may be used alone or in combination to predict the timing of the steps for the medical procedure to be performed by the medical personnel. In some embodiments, the timing of the steps may vary depending on an anatomy type of the patient. Machine learning may be useful for predicting the timing for each of a series of predicted steps for the procedure based on the collected information. For example, for Anatomy Type A, in the past Step 2 typically takes about 5 minutes while for Anatomy Type B, in the past Step 2 may typically take about 15 minutes. Optionally, medical personnel may or may not provide feedback in real-time whether the predicted timing of the steps are correct for the particular patient. In some embodiments, the feedback may be useful for improving step timing prediction in the future.

As medical personnel are performing a medical procedure, the various steps for a medical procedure may be recognized 460 using a machine learning algorithm. In some embodiments, video information, audio data, medical records, and/or inputs by medical personnel may be used alone or in combination to recognize the steps for the medical procedure that are being performed by the medical personnel. Machine learning may be useful for detecting and recognizing a series of steps for the procedure based on the collected information. Optionally, medical personnel may or may not provide feedback in real-time whether the detected steps are correct for the particular patient. In some embodiments, the feedback may be useful for improving step recognition in the future.

Similarly, during a medical procedure, the timing for the various steps for a medical procedure may be recognized 470 using a machine learning algorithm. In some embodiments, video information, audio data, medical records, and/or inputs by medical personnel may be used alone or in combination to recognize the timing of the steps for the medical procedure that are being performed by the medical personnel. For instance, the systems and methods provided herein may recognize the time at which various steps are started. The systems and methods provided herein may recognize a length of time it takes for the steps to be completed. The systems and methods provided herein may recognize when the next steps are taken. Machine learning may be useful for detecting and recognizing timing for a series of steps for the procedure based on the collected information. Optionally, medical personnel may or may not provide feedback in real-time whether the timing of the detected steps are correct for the particular patient. In some embodiments, the feedback may be useful for improving step timing recognition in the future.

Machine learning may be useful for additional steps, such as recognizing individuals at the location (e.g., medical personnel) and items (e.g., medical products, medical devices) being used. The systems and methods provided may be able to analyze and identify individuals in the room based on the video frames and/or audio captured. For example, facial recognition, motion recognition, gait recognition, voice recognition may be used to recognize individuals in the room. The machine learning may also be utilized to recognize actions taken by the individuals (e.g., picking up an instrument, medical procedure steps, movement within the location). The machine learning may be utilized to recognize a location of the individual.

In some embodiments, the machine learning may utilize deep convolution neural networks/Faster R-CNN Nast NasNet (COCO). The machine learning may utilize any type of convolutional neural network (CNN) and/or recurrent neural network (RNN). Shift invariant or space invariant neural networks (SIANN) may also be utilized. Image classification, object detection and object localization may be utilized. Any machine learning technique known or later developed in the art may be used. For instance, different types of neural networks may be used, such as Artificial Neural Net (ANN), Convolution Neural Net (CNN), Recurrent Neural Net (RNN), and/or their variants.

The machine learning utilized may optionally be a combination of CNN and RNN with temporal reference, as illustrated in FIG. 4B. Input, such as cameras images, external inputs, and/or medical inputs may be provided to a tool presence detection module. The tool presence detection module may communicate with EnodoNet. Training images may be provided for fine-tuning, which may provide data to EnodoNet. Additional input, such as camera images, external inputs, and medical images may be provided to EnodoNet. The output from EnodoNet may be provided to long short-term memory (LSTM). This may provide an output of a confidence score, phase/step recognition, and/or confusion matrix.

The machine learning may optionally utilize CNN for Multiview with sensors as illustrated in FIG. 4C. In some embodiments, inputs, such as various camera views/medical images with sensors, and/or external imaging with sensors may be provided to a CNN learning module. This may provide output to feature maps, which may in turn undergo Fourier feature fusion. The data may then be conveyed to a fully connected layer, and then be provided to Softmax, and then be conveyed as an output.

In some embodiments, the machine learning as described and applied herein may be an artificial neural network (ANN) as illustrated in FIG. 4D. The Multiview with sensors may be provided as illustrated. For instance, an input, such as one or more camera views/medical image or video with sensors may be provided to a predictive (computer vision/natural language processing) CV/NLP module. The output may be conveyed to an ANN module. The output from the ANN may be an analysis score or decision.

FIG. 4E shows an example of scene analysis utilizing machine learning, in accordance with embodiments of the invention. An input may comprise one or more camera views and/or medical image or video with sensors. The input may be provided to a module that may perform one or more functions, such as external input like vitals (e.g., ECG), tool detection, hand movement tracking, object detection and scene analysis, and/or audio transcription and analysis. The output from the module may be provided to a Markov logic network. Data from a knowledge base may also be provided to a Markov logic network. The output from the Markov logic network may be an output activity descriptor.

A location for a medical procedure, such as an operating room, may have one or more cameras which can recognize actors and instruments that are being used using deep convolution neural networks/Faster R-CNN Nast NasNet (COCO) where image classification, object detection, and/or object localization may occur. An audio enhancement module, such as a microphone array as described elsewhere herein, may also be provided at the location for the medical procedure, which can capture everything that is spoken and can convert text to speech for documentation. Using beamforming techniques, the systems ad methods provided can identify an individual that is speaking and the content of the speech. In situations where there is no speech, the systems and methods may rely on video/image data to generate documentation. In addition to storing data related to the entire medical procedure and documenting the procedure, the systems ad methods may be able to generate highlights for the documents and surgery which is composed of video and images.

Medical consoles may be installed on-site (e.g., surgery rooms) which may have multiple cameras and video/audio feeds along with all the skills and tools required to conduct a medical procedure. A separate video feed may be generated in real-time where the next steps that a medical practitioner should be doing along with analysis of the surgery which is going on. This may function as a surgery navigator for doctors. These instructions and video feed that is generated may be played slowly or quickly by adjusting context and scenario of the surgery room. The systems and methods may continuously learn new procedures, surgeries and continuously add data sets which can be used in following medical procedures. These data sets and intelligence may be shared across multiple medical consoles in real-time either through the cloud, P2P or P2P multicast. In addition, the systems and methods provided may be able to add context intelligence and data sets through the platform which can be used by these consoles in real-time.

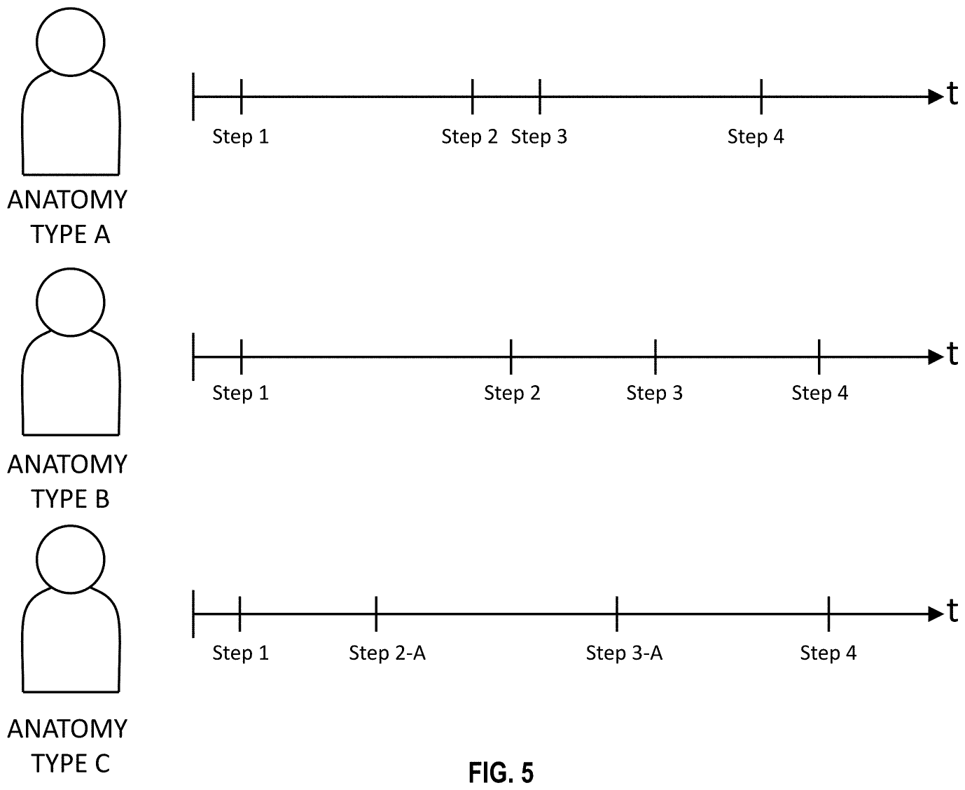

FIG. 5 shows an example of generating different predicted steps based on recognized anatomy type, in accordance with embodiments of the invention. As previously described, anatomy type for a patient may be recognize and/or provided by medical personnel. Anatomy type may differ based on the patient's gender, age, weight, height, placement of anatomical features, size of anatomical features, distances between anatomical features, presence of scar tissues, medical history, pre-existing conditions, health, or other factors, including any others described elsewhere herein. For example, Anatomy Type A, Anatomy Type B, and Anatomy Type C may be recognized and distinguishable from one another. Any number of anatomy types may be provided to any degree of specificity. In some embodiments, the number of available anatomy types may depend on the type of medical procedure. For example, different number of anatomy types for consideration may be provided for hand surgery compared to neurosurgery. Any number of anatomy types may be provided. For instance, 1 or more, 2 or more, 3 or more, 4 or more, 5 or more, 7 or more, 10 or more, 15 or more, 20 or more, 30 or more, or 50 or more anatomy types may be possible for a given medical procedure. The factors used to determine the anatomy types may depend on the type of medical procedure. For example, the factors used to distinguish between patient anatomy types may be different for hand surgery as compared to neurosurgery. The factors used to distinguish patient anatomy types may include factors that are specific to the region at which the surgery is being performed.

A series of predicted steps may be provided for a given medical procedure. In some embodiments, the steps provided and/or the predicted timing of the steps may differ based on anatomy type.

For example, the same steps may be provided for a given medical procedure for different anatomy types. In one instance, for Anatomy Type A, the medical procedure may include Step 1, Step 2, Step, 3, and Step 4. As illustrated, they may occur at particular instances in time (t). For Anatomy Type B, the same type of medical procedure may also include the same steps, Step 1, Step 2, Step 3 and Step 4. However, the predicted timing for these steps may vary for the different anatomical types. For example, particular anatomies may make certain steps within the procedure more difficult, which may result in more time being taken for that particular step. For instance, Step 1 may take longer with Anatomy Type B than Anatomy Type A. Similarly, Step 2 may take longer with Anatomy Type B, than Anatomy Type A. However, Step 4 may take about the same amount of time regardless of whether the patient is of Anatomy Type A or Anatomy Type B.

In some embodiments, different steps may be provided for a given medical procedure for different anatomy types. In one instance, for Anatomy Type A, the medical procedure may include Step 1, Step 2, Step, 3, and Step 4. As illustrated, they may occur at particular instances in time (t). For Anatomy Type C, the same type of medical procedure may include one or more different steps, Step 2-A and Step 3-A. For instance, for different anatomy types different techniques or steps may be utilized to accommodate the different anatomies. The different techniques and/or steps may be recommended as having the best outcome and/or being performed in the most efficient manner. In some instances, one or more of the steps may be the same (e.g., Step 1, and Step 4). The predicted timing for these steps may vary for the different anatomical types. For example, particular anatomies may make certain steps within the procedure more difficult, which may result in more time being taken for that particular step. For instance, Step 1 may take longer with Anatomy Type A than Anatomy Type C. Different step types may result in different timings. For example, Step 2-A may take longer with Anatomy Type C, than Step 2 with Anatomy Type A. In some instances, some of the steps (whether the same steps, or different steps at equivalent stages) may have the same predicted duration.

In some instances, the timing and/or selection of steps may be based on large data sets. There may be acceptable variation to the timing and/or steps selected. The predictions may be based on the mean or median timings and/or the most commonly selected steps (or the steps with the best outcome, and/or steps that were able to be performed most efficiently with an acceptable outcome). In some embodiments, the predicted timing and/or selection of steps may change over time as the data sets evolve and new techniques emerge in the field. The predicted timing may depend on actual past times to perform particular procedures for the same or similar circumstances, such as the same or similar anatomy types. In some instances, other circumstances may be taken into account when generating predicted timing, such as expertise or experience of practitioner, location, facility, type of surgical procedure, type of instruments expected to be used, or any other factors. In some embodiments, the predicted time may be generated with aid of one or more machine learning models that may or may not utilize actual previous measured data as training data sets, or may utilize generated data as training data sets.

Optionally, medical personnel may view the predicted steps and/or timing before performing the procedure and/or in real-time while performing the procedure. Alternatively, the medical personnel need not view the steps and may perform the procedure according to his or her own experience.

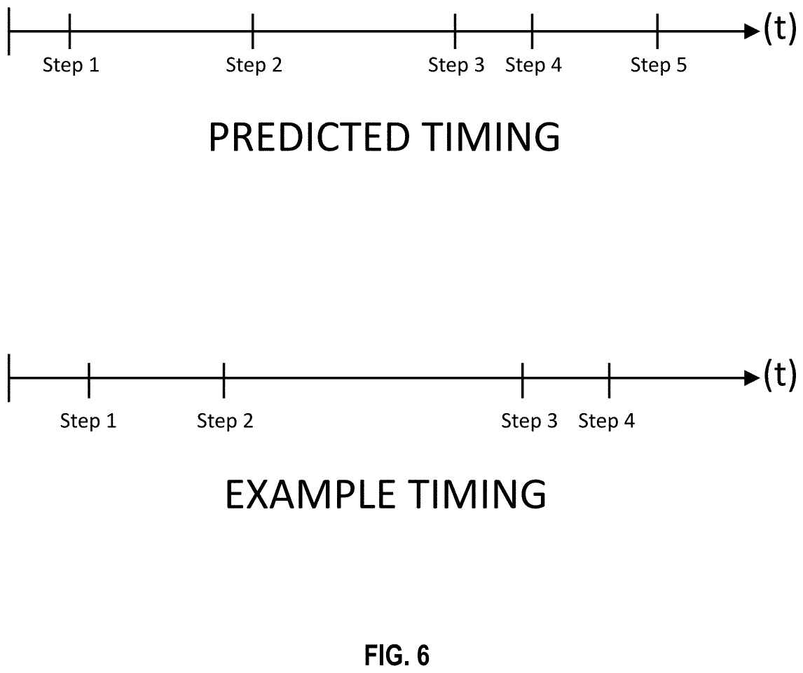

FIG. 6 shows an example of a comparison between predicted steps for a procedure being compared with steps as they actually occur in real-time, in accordance with embodiments of the invention. For instance, the video analysis system may generate a set of predicted timing for various steps to perform a medical procedure. As previously described, the steps and associated timing may be predicted based on a patient anatomy type. Alternatively, the steps and/or timing may be provided without being based on a patient anatomy type. In some instances, a set of steps for a particular medical procedure may be set and the timing may be predicted based on patient anatomy type.

As illustrated, the various steps (e.g., Step 1, Step 2, Step 3, Step 4, and Step 5) may have various predicted lengths of time for the medical personnel to complete. The steps may have various predicted lengths. For instance, Step 3 may be predicted to take less time than Step 2.

While the medical personnel is performing the medical procedure (Example Timing), the steps performed by the medical personnel may be recognized. The timing associated with the performance of the steps may be recognized. In some embodiments, machine vision techniques may be used to recognize the steps being performed. In some instances, the analysis of the video may occur within the context of the predicted steps to be performed. Images from video frames, audio data, medical records, information input by medical personnel or other sources may be used alone or in combination to recognize steps that are occurring.

In some instances, anatomical features may be recognized and detected to aid in recognizing the steps being performed. For instance, the portion of the patient's body, such as the various specific features within the portion of the patient's body may be useful to detect the steps being performed. The medical devices and/or products used may be recognized from video images, or based on medical personnel input, and may be useful for recognizing a step that is being performed. For particular steps, certain types of medical devices or tools may be required, and the identity of the tools and/or devices used may be useful in detecting the steps being performed. In some instances, audio information may be useful for detecting steps as well. For instance, medical personnel may announce the step that he or she is about to perform before taking the step, or while performing the step. In some instances, medical personnel may dictate their actions are they are occurring. Optionally, the medical personnel may ask for assistance or tools from other medical personnel and this may provide information that may be useful for detecting the steps taken. In some instances, a patient's medical records may be a useful aid in determining the steps being taken. This may be useful in eliminating steps that may be not be possible or desirable for a particular patient.

As the steps are being performed, their timing may be recorded and measured. For instance, as the video analysis system detects each step is starting, the system may make note of the time at which each step is occurring.

The timing may be provided to any degree of desired accuracy and/or precision. For instance, the timing may be provided based on the order of 10 minutes or less, 5 minutes or less, 3 minutes or less, 1 minute or less, 30 seconds or less, 20 seconds or less, 15 seconds or less, 10 seconds or less, 5 seconds or less, 3 seconds or less, 1 second or less, 0.5 seconds or less, 0.1 seconds or less, 0.05 seconds or less, or 0.01 seconds or less, or any greater degree of precision.

The predicted timing may be compared with the actual (example) timing for the various steps as they occur in real-time. When a significant disparity in timing exists, the disparity may be flagged. In some instances, a notification may be provided to a medical personnel in real-time while they are performing the procedure. For instance, a visual notification or audio notification may be provided when the disparity has been detected. In some embodiments, a disparity may need to reach a threshold in order to be flagged. The threshold for the disparity may be set ahead of time. The threshold may be set based on an absolute value (e.g., number of minutes, seconds, etc.) and/or relative value (e.g., percentage of the predicted time for the step). In some instances, the threshold value may depend on the standard deviation from the various data sets collected. For example, if a wider variation in timing is provided through the various data sets, then a greater threshold or tolerance may be provided. The threshold value may be fixed or may be adjustable. In some embodiments, the medical personnel, or another individual at a health care facility (e.g., colleague, supervisor, administrator) may set the value. In some embodiments, a single threshold may be provided. Alternatively, multiple levels of thresholds may be provided. The multiple threshold levels may be useful in determining a degree of disparity and may result in different types of actions or notifications to the medical personnel.

As illustrated in the example, Step 1 may have a particular predicted timing, and Step 1 may actually be performed within approximately the same amount of time as predicted. This may cause no flags to be raised. In another example, Step 2 may be expected to occur within a particular length of time, but in reality may actually take a significantly longer period of time. When a significant deviation occurs, this difference may be flagged. This may allow medical personnel to later review this disparity and figure out why the step took longer than expected. This may be useful for introducing new techniques or providing feedback to the medical personnel on how the medical personnel may be able to perform more efficiently in the future.

In some instances, a medical personnel may be able to perform a step faster than predicted. When this occurs, it may be useful information to provide for educational purposes to other medical personnel. This information may be flagged as a useful teaching opportunity to other medical personnel.

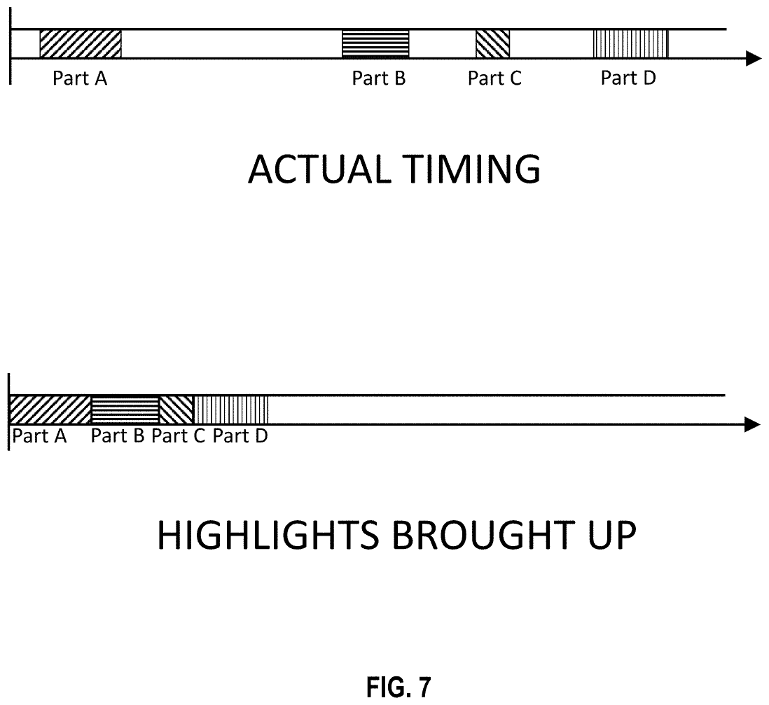

FIG. 7 shows an example of relevant portion of video being identified and brought up for viewing, in accordance with embodiments of the invention.

The video may be useful in providing notifications to medical personnel in real-time while the procedure is occurring. The video may also be useful for providing information to the medical personnel after a procedure has been completed. The medical personnel may wish to review the procedure after the procedure has been completed. This may occur immediately after the procedure is completed, or at a later time. In some embodiments, other individuals may wish to review the procedure as well. For instance, a colleague, supervisor, and/or administrator may be able to review the procedure as well.

As procedures can be lengthy, reviewing an entirety of a video in its raw form may take up a large amount of unnecessary time. It may be desirable to provide a simplified method of showing a user relevant portions of the video only, thus saving time. In some embodiments, one or more relevant portions of a video may be identified (e.g., Part A, Part B, Part C, Part D).

In some embodiments, a user may manually identify the relevant portions in real-time during a surgical procedure. For instance, the user may make a verbal command, provide a gesture-based input, or provide a direct input into a touchscreen that denotes that a particular portion of the procedure is relevant and should be flagged.

Optionally, the video analysis system may automatically determine one or more relevant portions in real-time during a surgical procedure. The video analysis system may automatically make the determination with aid of one or more processors and without requiring user input. For instance, when a step that is performed is actually different from a predicted step, the portion of the video corresponding to the performed step may automatically be flagged as being relevant. When a step that is performed takes a significantly different length of time compared to a predicted length of time for that step, the portion of the video corresponding to the performed step may automatically be flagged as being relevant. When the difference in timing between the predicted amount of time for the step and the actual amount of time taken for the step exceeds a threshold, as described elsewhere herein, the portion of the video corresponding to the step may automatically be flagged as relevant.

For instance, it may be desirable to flag a step as relevant when it takes much longer than predicted. A medical personnel or other individual may wish to review the step and determine why it took so much longer than predicted. In some instances, a step taking longer may be indicative of an event or issue that arose that required more time for the medical personnel to perform the step. In some instances, the step taking longer may indicate that the medical personnel is not using the most efficient technique, or is having difficulty with a particular step, which may be helpful to provide additional review.

In some instances, it may be desirable to flag a step as relevant when it takes significantly less time than predicted. A medical personnel or other individual may wish to review the step and see how the medical personnel was able to save time on a particular step. This may provide a useful teaching opportunity to other individuals that may wish to mimic a similar technique. This may also provide a recognition of a particular skillset that the medical personnel may have.

In some instances, the video analysis system may automatically determine one or more relevant portions based on other factors. For example, image analysis may be used to detect incidents of note or complications that arose during a medical procedure. Such corresponding portions may be flagged as relevant.

The portions of the video that have been flagged as relevant may be brought up to the front of the video as part of the ‘highlights’. The highlights section may be provided separately from the rest of the video, or may play before the entirety of the video. The highlights section may comprise the portions that have been flagged as relevant. The highlights section may provide the relevant portions in the order that they occurred. This may allow the medical personnel and/or other individuals to save time by watching the most relevant portions of the video. The relevant portions of the video may be automatically flagged and brought up, thus saving time.

In some embodiments, a single camera may be used to capture the images that are being analyzed. In some instances, multiple cameras may be used to capture the images that are being analyzed. The multiple cameras may capture images simultaneously. In some instances, the images from the multiple cameras may be provided as a side by side, or array view. The images from the multiple cameras may be shown simultaneously on the same screen. When a portion of a video is flagged as relevant, the images from all of the cameras that were captured at the same time may be brought up and shown together. In other instances, only the video from the camera that has been flagged as relevant may be brought up and shown. In some embodiments, the video analysis system may select the camera that provides the best view of the procedure for a given time period that has been flagged as relevant. This may be the same camera that provided the video that has been flagged as relevant, or another camera.

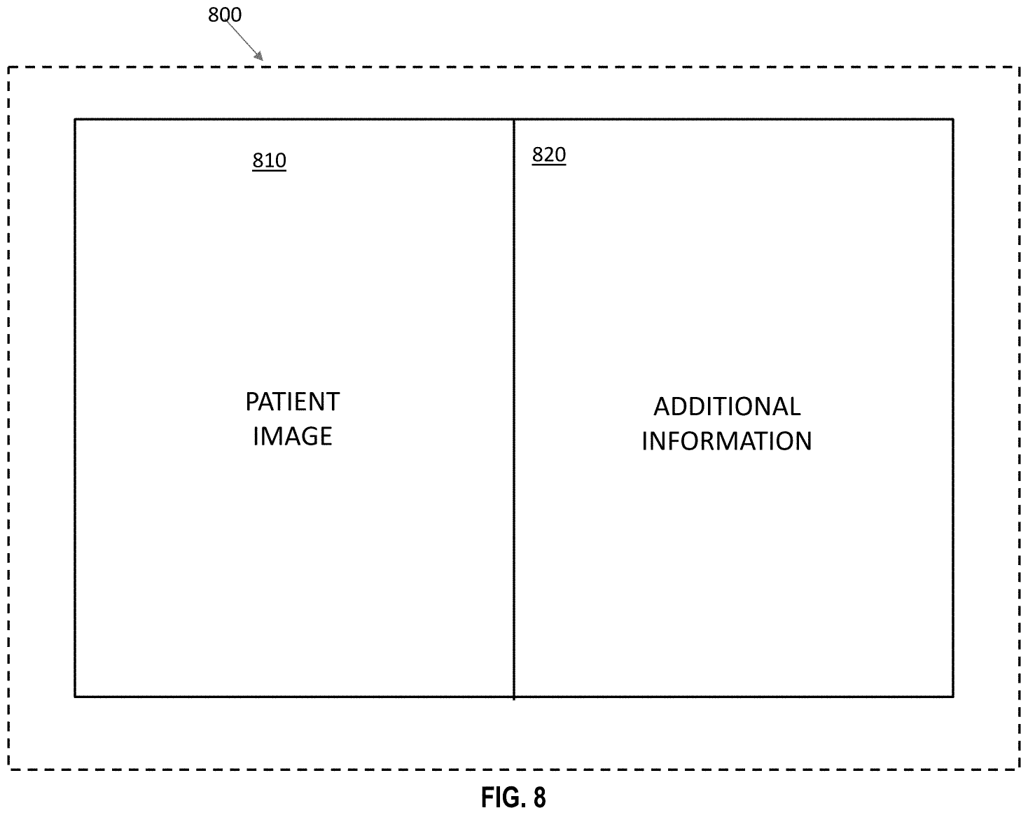

FIG. 8 shows an example of a user interface 800 that may display images captured by one or more video cameras and provide support to a medical practitioner in real-time, in accordance with embodiments of the invention. The user interface may be displayed on a communication device such as a local communication device on a medical console. For instance, the user interface may be displayed on a screen of a device at the location of the medical personnel performing the procedure. The user interface may be displayed on a screen of a remote communication device.

A user interface may include a display of one or more patient images 810. The one or more patient images may be captured with aid of one more cameras at the location of the patient. As previously described, one or more cameras on a medical console or off a medical console may be utilized to capture images of the patient or provide any other relevant images.

In some instances, a single image may be displayed to medical personnel at a given moment. The medical personnel may toggle between different views from different cameras. The images may be displayed in a sequential manner.

In some other instances, multiple images may be simultaneously displayed to medical personnel at a given moment. For example, multiple images may be displayed in a row, in a column, and in/or in an array. Images may be simultaneously displayed in a side-by-side manner. In some instances, smaller images may be inserted within a bigger image. A user may select the smaller image to expand the smaller image and shrink the larger image. Any number of images may be displayed simultaneously. For instance two or more, three or more, four or more, five or more, six or more, eight or more, ten or more, or twenty or more images may be displayed simultaneously. The various displayed images may include images from different cameras. The cameras may be live-streaming to different regions simultaneously.

In some instances, images may also include images from auxiliary devices. For instance, one or more auxiliary devices may be connected to a medical console (e.g., plugged into a medical console). Images relating to the auxiliary device may be displayed along with images from cameras. In some instances, the auxiliary devices may include ECG monitors, endoscopes, laparoscopes, or any other type of device.

The user interface may include a display of additional information 820. The additional information may relate to a procedure being performed or about to be performed by medical personnel. The additional information may include steps relating to the medical procedure. For example, a list of steps predicted in order to perform the medical procedure may be displayed. The list of steps may be presented in a chronological order with the first step appearing at the top of the list. In some embodiments, a single list of steps may be presented. In some embodiments, the lists may have sub-lists and so forth. For instance, the lists may be appear in a nested fashion, where a step may correspond to a second list having details of how to perform each step. Any number of layers of lists and sub-lists for each step may be presented.

In some embodiments, the video analysis system may be capable of recognizing when medical personnel has performed each step. In some embodiments, when the system detects that the medical personnel has performed a step (or sub-step), the additional information may be updated. Any description herein of a step may apply to any level of sub-step. For instance, as each step is completed, a checkmark or other type of visual indicator may be provided that may visually distinguish a completed step. In some instances, a completed step may visually disappear from the additional information section. In other embodiments, medical personnel may provide an input that indicates when a step has been completed.

Updating a step list in real-time may assist medical personnel in tracking progress throughout the medical procedure. In some embodiments, a visual indicator (e.g., checkmark, highlight, different color, icon, strikethrough, underline) may be provided to visually differentiate completed steps from steps that have not yet been completed. In some instances, detected steps or conditions during the medical procedure may cause the predicted or recommended steps to change. The video analysis system may automatically detect when such a condition has occurred. For example, if the medical personnel accidentally cuts an artery, the steps may be updated to provide recommendations on how to deal with the cut artery first before moving on with the rest of the procedure. The steps may be updated in real-time.

The predicted steps of the medical procedure may be generated based on anatomy of the patient, as described elsewhere herein. In some embodiments, the predicted steps for the medical procedure may be generated based on context, such as available or detected instruments, information from scene-awareness, the identity of the medical personnel performing the procedure, available remote support, real-time condition of the patient, or any other factors. The predicted steps may utilize information from a large data set and may be updated in real-time based on real-time updates to the data set. The predicted steps may recognize updates relating to the condition of the patient or new information gathered about the patient, and may be updated in real-time accordingly.

The predicted steps may utilize machine learning which may continuously learn new procedures, surgeries, and/or add new datasets which can be used in following surgeries. For instance, if new techniques are emerging in the data sets as being effective, the predicted steps may be updated to utilize the new techniques. The predicted steps may function like a surgery navigator for the medical personnel, and may aid the medical personnel in learning new procedures and performing the instant procedure.

The datasets that may be used to update the predicted steps may utilize information from multiple medical consoles. The multiple medical consoles may be at the same health care facility or may be at different healthcare facilities. The data sets and/or intelligence from the datasets may be shared across all medical consoles (within the health care facility or across multiple health care facilities) in real-time. The sharing may occur utilizing a cloud-computing infrastructure, or utilizing peer-to-peer (P2P) communications. The systems and methods provided herein may utilize any type of infrastructure, such as P2P, Client/Server, Hybrid (P2P Multicast+Client Sever), P2P Multicast, Simulcast, Multicast, with help of HTTP ABR streaming protocols in addition to real time streaming protocols as a few different ways to stream data.

In some instances, the additional information may include timing information. For example, expected timing for each step may be displayed in a visually mapped manner with each step. For example, for predicted steps, the predicted amount of time for each step may be displayed. For completed steps, the amount of time to complete the step may be displayed. In some instances, for completed steps, the amount of time to complete the step may be displayed along with the predicted time to conduct the step. A comparison for the predicted time vs. the actual amount of time to complete the step may be presented. In some embodiments, the comparison may be provided as numbers, fractions, percentages, visual bars, icons, colors, line graphs, or any other type of comparison.

The timing information may include a total timing information. For instance, alternative to or in addition to showing timing information for each step, the overall timing information or progress may be displayed. For instance, a total amount of time that the medical personnel is lagging, or ahead of the predicted time, may be displayed. In some instances, the total amount of time may be displayed as a numerical time value (e.g., hours, minutes, seconds), or as a relative value (e.g., percentage of predicted time or actual time, etc.). In some instances, a visual display, such as a bar may be provided. The visual display may include a bar representing a timeline. The bar may show a total predicted time to complete the medical procedure. The predicted breakdown of times at each step may or may not be shown on the bar. The medical personnel's current amount of time spent may be shown relative to the bar. An updated predicted amount of time to complete the medical procedure may also be displayed as a second bar or overlap with the first bar. Overall timing or progress information may be provided to the medical personnel in a visual manner.

The additional information may include any type of helpful information to the medical personnel as the medical personnel performs the procedure. The medical console may function as a smart console that may provide assistance to the medical personnel. In some instances, the additional information may include medical records relating to the patient. The systems and methods provided may automatically interface with electronic medical records (EMR) for the patient and pull relevant information as the medical personnel is performing the procedure. The smart console may listen continuously with automatic speech recognition (ASR) and pull up relevant details from a database of documents. In some embodiments, an audio enhancement module may be used to assist in collecting verbal commands. The smart console may pull up information relevant to queries or requests by medical personnel in real-time. Any voice recognition techniques known or later developed in the art may be utilized.

In some embodiments, information helpful to the medical personnel may include videos or instructions on how to perform certain steps. For instance, if a medical personnel is having difficulty with a particular step, the medical personnel may request a training video or a series of instructions to walk through the step. In some instances, images may be used to aid the medical personnel in walking through the step. If the medical personnel is having difficulty using a medical device or product, the medical personnel may verbally request help from a vendor representative, as described elsewhere herein. In some instances, if the medical personnel is having difficulty using a medical device or product, the medical personnel may request a training video or series of instructions to walk through use of the device or product.

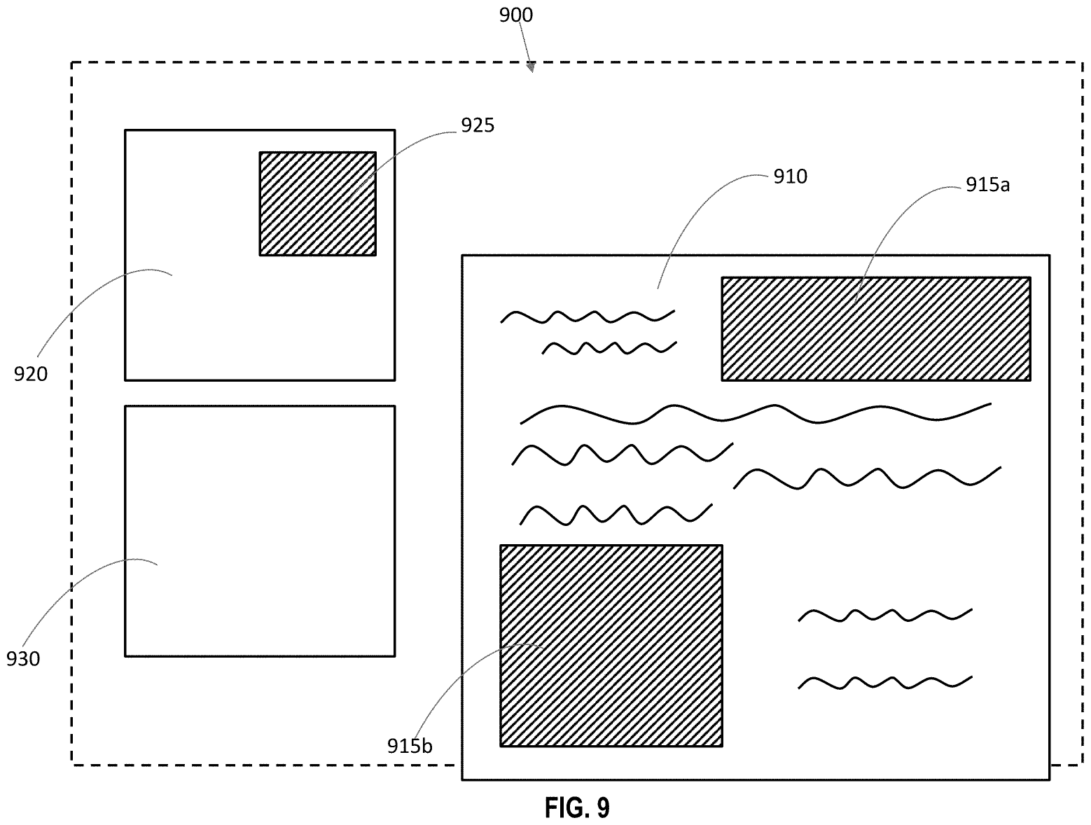

FIG. 9 shows an example of a user interface 900 when personal data is removed, in accordance with embodiments of the invention. In some embodiments, images captured by the videos may include personal information relating to the patient. For instance, a chart or document may have a patient's name, birth date, social security number, address, telephone number, insurance information, or any other type of personal information. In some embodiments, it may be desirable to redact personal information for the patient from the video. In some instances, it may be desirable to anonymize information shown on the video in order to comply with one or more set of rules, procedures or laws. In some instances, all information shown on the video may be compliant with the Health Insurance Portability and Accountability Act (HIPAA-compliant).

As illustrated, the user interface may show information relating to a patient, such as a chart or a set of medical records 910. The chart or medical records may include a physical document and/or electronic documents that have been accessed during the procedure. The information may include personal or sensitive information relating to the patient. Such information may be automatically identified by the video analysis system. The video analysis system may use object and/or character recognition to be able to identify information displayed. In some instances, word recognition techniques may be used to analyze the information. Natural language processing (NLP) algorithms may optionally be used. In some instances, when personal information is identified, the personal information may be automatically removed 915a, 915b. Any description herein of personal information may include any sensitive information relating to the patient, or any information that may identify or provide personal characteristics of the patient.

The user interface may include one or more images 920, 930. The images may include video captured by one or more cameras as described elsewhere herein. The images may include data or images from auxiliary devices as described elsewhere herein. In some instances, one or more of the images may include personal information that may need to be removed 925. In some instances, an identifying characteristic on a patient may be captured by a video camera (e.g., the patient's face, medical bracelet, etc.). The one or more images may be analyzed to automatically detect when the identifying characteristic is captured within the image and remove the identifying characteristic. In some instances, object recognition may be used to identify personal information. For instance, recognition of an individual's face or medical bracelet may be employed in order to identify personal information that is to be removed. In some instances, a patient's chart or medical records may be captured by the video camera. The personal information on the patient's chart or medical records may be automatically detected and removed.

The personal information may be removed by being redacted, deleted, covered, obfuscated, or using any other techniques that may conceal the personal information. In some instances, the systems and methods provided herein may be able to identify the size and/or shape of the information displayed that needs to be removed. A corresponding size and/or shape of the redaction may be provided. In some instances, a mask may be provided over the image to cover the personal information. The mask may have the corresponding shape and/or size.

Accordingly, any video that is recorded and/or displayed may anonymize the personal information of the patient. In some instances, the video that is displayed at the location of the medical personnel (e.g., within the operating room) may show all of the information without redacting the personal information in real-time. Alternatively, the video that is displayed at the location of the medical personnel may have the personal information removed.

In some instances, the video may be displayed to one or more individual outside the location of the medical personnel (e.g., outside the operating room, or outside the health care facility). In some instances, the video may be displayed to one or more individuals (e.g., other medical practitioners, vendor representatives) that may be providing support to the medical procedure remotely. In some instances, the video may be broadcast to a number of individuals that may be viewing the procedure. The individuals may be viewing the procedure for training or evaluation purposes. The video as live-streamed to the one or more individuals may automatically have the data anonymized. The personal information may be removed in real-time so that no individual outside the operating room views any personal information of the individual.

Optionally, the video may be viewed and played back at a later time. In some instances, when the video is provided at a later time, the personal information may automatically be removed and/or anonymized.

The video analysis system may optionally employ smart translations. The smart translations may build therapy-specific language models that may be used to buttress various language translation with domain specific language. For instance, for particular types of procedures or medical areas, various vernacular may be used. Different medical personnel may use different terms for the same meaning. The systems and methods provided herein may be able to recognize the different terms used and normalize the language.

This may apply to commands spoken by medical personnel during a medical procedure. The medical personnel may ask for support or provide other verbal commands. The medical console or other devices may use the smart translations. This may help the medical console and other devices recognize commands provided by the medical personnel, even if the language is not standard.

In some instances, a transcript of the procedure may be formed. One or microphones, such as an audio enhancement module, may be used to collect audio. One or more members of the medical team may speak during the procedure. In some instances, this may include language that relates to the procedure. The smart translations may automatically include translations of terminology used in order to conform to the medical practice. For instance, for certain procedures, certain standard terms may be used. Even if the medical personnel use different terms, the transcript may reference the standard terminology. In some embodiments, the transcript may include both the original language as well as the translations.

In some instances, when individuals are speaking with one another via one or more communication devices, the smart translations may automatically offer up the standard terminology as needed. If one user is speaking or typing to another user and utilizing non-standard terminology, the smart translations may automatically conform the language to standard terminology. In some instances, each medical area or specialty may have its own set of standard terminology. Standard terminology may be provided within the context of a procedure being conducted.

Optionally, the systems and methods provided herein may support multiple languages. For example, an operating room may be located within the United States with the medical personnel speaking English. An individual providing remote support may be located in Germany and may speak German. The systems and methods provided herein may translate between different languages. The smart translations may be employed so that the standard terminology is used in each language. Even if different words or phrasing is used by the individuals, the smart technology may make sure the words that are translated conform to the standard terminology in each language with respect to the medical procedure.

The smart translations may be supported locally at a medical console. The smart translations may occur on-board the medical console. Alternatively, the smart translations may occur at one or more remote servers. The smart translations may be implemented through a cloud computing infrastructure. For instance, the smart translations may occur in the cloud and be pushed back to the relevant consoles.

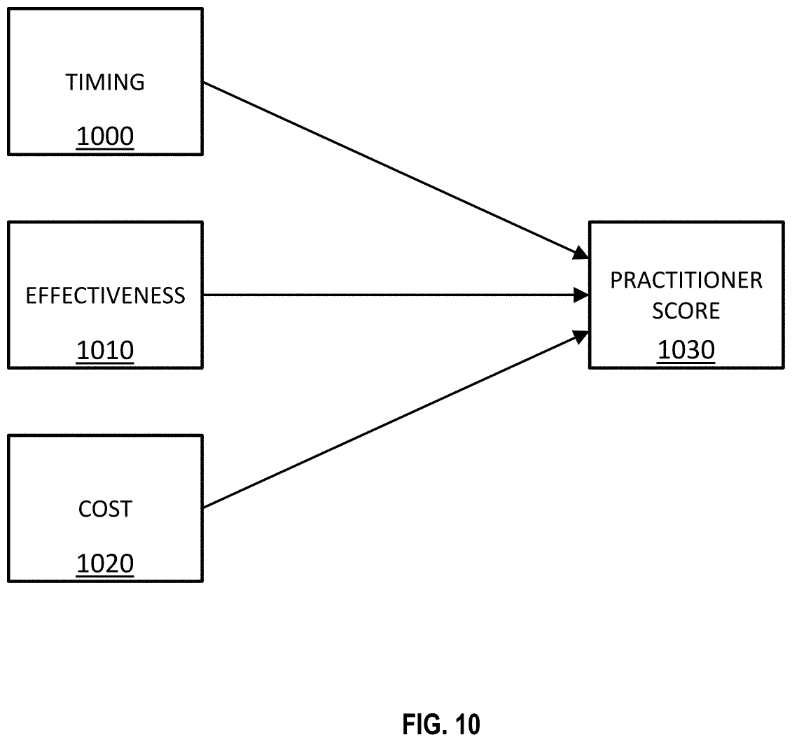

FIG. 10 shows an example of factors that may be used to assess medical personnel performance, in accordance with embodiments of the invention. In some embodiments, it may be desirable to assess medical personnel performance after a procedure has been completed. This may be useful as feedback to the medical personnel. This may allow the medical personnel to focus on improving in areas as needed. The medical personal may wish to know his or her own strengths and weaknesses. The medical personnel may wish to find ways to improve his or her own effectiveness and efficiency.

In some embodiments, it may be desirable for other individuals to assess medical personnel performance. For instance, a health care facility administrator, or a medical personnel's colleague or supervisor may wish to assess the performance of the medical personnel. In some embodiments, medical personnel performance assessment may be useful for assessing the individual medical personnel, or a particular group or department may be assessed as an aggregate of the individual members. Similarly, a health care facility or practice may be assessed as an aggregate of the individual members.

The medical personnel may be assessed in any manner. In one example, the medical personnel may be given a score 1030 for a particular medical procedure. The score may be a numerical value, a letter grade, a qualitative assessment, a quantitative assessment, or any other type of measure of the medical personnel's performance. Any description herein of a score may apply to any other type of assessment.

The practitioner's score may be based on one or more factors. For instance, timing 1000 may be provided as a factor in assessing practitioner performance. For instance, if the medical personnel is taking much longer than expected to perform medical procedures, or certain steps of medical procedures, this may reflect detrimentally on the medical personnel's assessment. If the medical personnel has a large or significant deviation from expected time to completion for a medical procedure, this may detrimentally affect his or her score. Similarly, if the medical personnel takes less time than expected to perform the medical procedure, or certain steps of medical procedure, which may positively affect his or her assessment. In some instances, threshold values may be provided before the deviation is significant enough to affect his or her score positively or negatively. In some instances, the greater the deviation, the more that the timing affects his or her score. For example, if a medical personnel's time to complete a procedure is 30 minutes over the expected time, this may impact his score more negatively than if the medical personnel's time to complete the procedure is 10 minutes over the expected time. Similarly, if the medical personnel completes a procedure 30 minutes early, this may impact his score more positively than if the medical personnel's time to complete the procedure is 5 minutes under the expected time.

Other factors may be used to assess medical personnel performance. For instance, the effectiveness 1020 or outcome of the procedure may be a factor that affects the medical personnel's assessment. If complications arise, or if the medical personnel makes a mistake, this may negatively affect the medical personnel's score. Similarly, if the medical personnel has a complication-free procedure, this may positively affect the medical personnel's score. In some instances, recovery of the patient may be taken into account when assessing the performance of the medical personnel.

Another factor that may be taken into account is cost. For example, if the medical personnel uses more medical products or devices than expected, then this may add to the cost, and may negatively affect the medical personnel's assessment. For instance, if the medical personnel regularly drops objects, this may reflect detrimentally on the medical personnel's assessment. Similarly, if the medical personnel uses more resources (e.g., devices, products, medication, instruments, etc.) than expected, the cost may go up. Similarly, if the procedure takes longer than expected, the corresponding costs may also go up.

The systems and methods provided here may provide access to a large data set. This may allow for the medical personnel's performance to be compared relative to a large pool of individuals. This may provide a more accurate sense of where medical personnel can improve, or identifying areas of strength for the medical personnel. The video analysis system may be able to access information from many health care facilities in order to predict steps and/or timing, or outcomes of procedures. The medical personnel performance may be assessed relative to the entire population from whom data is collected. Optionally, the medical personnel performance may be assessed relative to the rest of the health care facility, the medical personnel's group or department.

The scoring of the medical personnel may take into account various elements of scene-awareness. For instance, the analyzed video may include information about the context and circumstances under which the medical personnel is performing the procedure. This context and circumstances may be taken into account when determining the score of the medical personnel. For example, if an emergency occurred within the building, and the medical personnel took a longer time to perform the step, the longer time may not be detrimental to the score of the medical personnel within the context at which the medical personnel was operating. Machine learning techniques, as described elsewhere herein may be used to provide scene-awareness and aid in evaluating the performance of the medical personnel. The machine learning techniques may be able to analyze the context based on which circumstances are outside of the control of the medical personnel and which are within the control of the medical personnel. Such different types of circumstances may be distinguished from one another.

In some embodiments, medical personnel may communicate with one or more remote individuals during a procedure. In some instances, the remote individual may be a vendor representative, another medical practitioner, a social worker, or any other individual.

The medical personnel may use a medical product that is supported by a vendor representative. The video analysis system may allow a vendor to provide a remote telepresence during a medical procedure, and support the medical product as needed. The medical personnel may consult with one or more additional medical practitioners. For instance, the video analysis system may enable a medical practitioner to provide a remote telepresence during the medical procedure. Any description herein of a remote individual may apply to any type of individual with whom the medical personnel may communicate.

Any description herein of a remote individual may apply to any other type of individual that may provide support during a medical procedure. For instance, any description herein of a remote individual may also apply to vendor representatives, outside medical professionals or specialists, consultants, technicians, manufacturers, financial support, social workers, or any other individuals.

Medical products may be provided by one or more vendors. Typically, vendors may make arrangements with health care facilities to provide medical products. Vendors may be entities, such as companies, that manufacture and/or distribute medical products. The vendors may have representatives that may be able to provide support to personnel using the medical devices. The vendor representatives (who may also be known as product specialists or device reps), may be knowledgeable about one or more particular medical products. Vendor representatives may aid medical personnel (e.g., surgeons, surgical assistants, physicians, nurses) with any questions they may have about the medical products. Vendor representatives may aid in selection of sizing or different models of particular medical products. Vendor representatives may aid in function of medical products. Vendor representatives may help a medical personnel use product, or troubleshoot any issues that may arise. These questions may arise in real-time as the medical personnel are using a product. For instance, questions may arise about a medical product while a surgeon is in an operating room to perform a surgery. Traditionally, medical representatives have been located at the first location with the medical personnel. However, this can be time consuming since the medical representative will need to travel to the location of the medical procedure. Secondly, the medical representative may be present but the medical representative's help may not always be needed, or may be needed for a very limited time. Then, the medical representative may have to travel to another location. It may be advantageous for a medical representative to communicate remotely as needed with personnel at the first location.

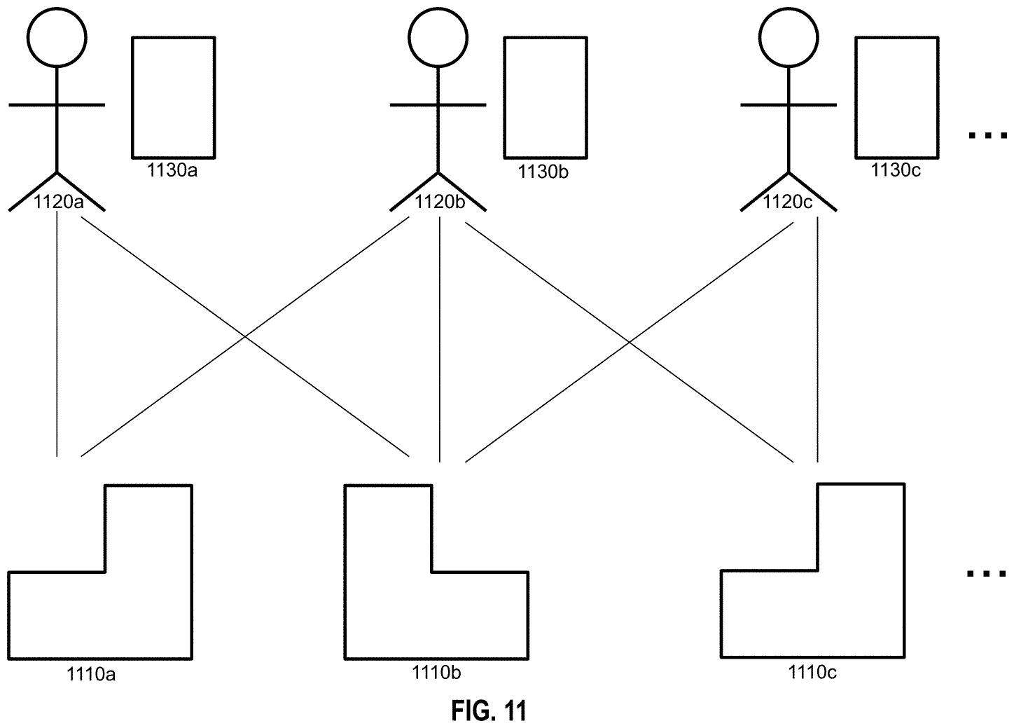

FIG. 11 shows an example of a system where multiple remote individuals may be capable of communicating with multiple health care facilities, in accordance with embodiments of the invention.

Various health care facilities 1110a, 1110b, 1110c may be provided. The health care facilities may be of the same or different type. For example, the health care facilities may include hospitals.

One or more vendors may provide medical products to the various health care facilities. For instance, a first vendor company may provide one or more types of medical products to a first health facility and a second health care facility. A second vendor company may provide one or more types of medical products to a first health facility and a second health care facility. A vendor company may provide one or more vendor representatives 1120a, 1120b, 1120c that may provide support for various medical products. The vendor representatives may provide support to one or more multiple health care facilities. The vendor representatives may utilize respective remote communication devices 1130a, 1130b, 1130c. Any description herein of vendor representatives may apply to any other type of remote individuals. Any description herein of medical products may apply to any type of services provided by the remote individuals, such as support and consulting services.

As illustrated, various remote individuals 1120a, 1120b, 1120c may provide support to various health care facilities 1110a, 1110b, 1110c. Such support may overlap with other remote individuals, or may not overlap. Such support may overlap for similar products or different products, or for similar medical procedures or different medical procedures. As illustrated, when remote individuals are providing support to multiple different health care facilities, allowing a remote individual to provide support remotely may advantageously save the remote individual a large amount of time.

To further improve the efficiency and ease for remote individual to communicate with the appropriate medical personnel and lend support, the systems and methods are provided herein that facilitate the communication. The health care facilities and the remote individuals may utilize systems and methods as provided herein to utilize the facilitated communication that allows for remote individual support.

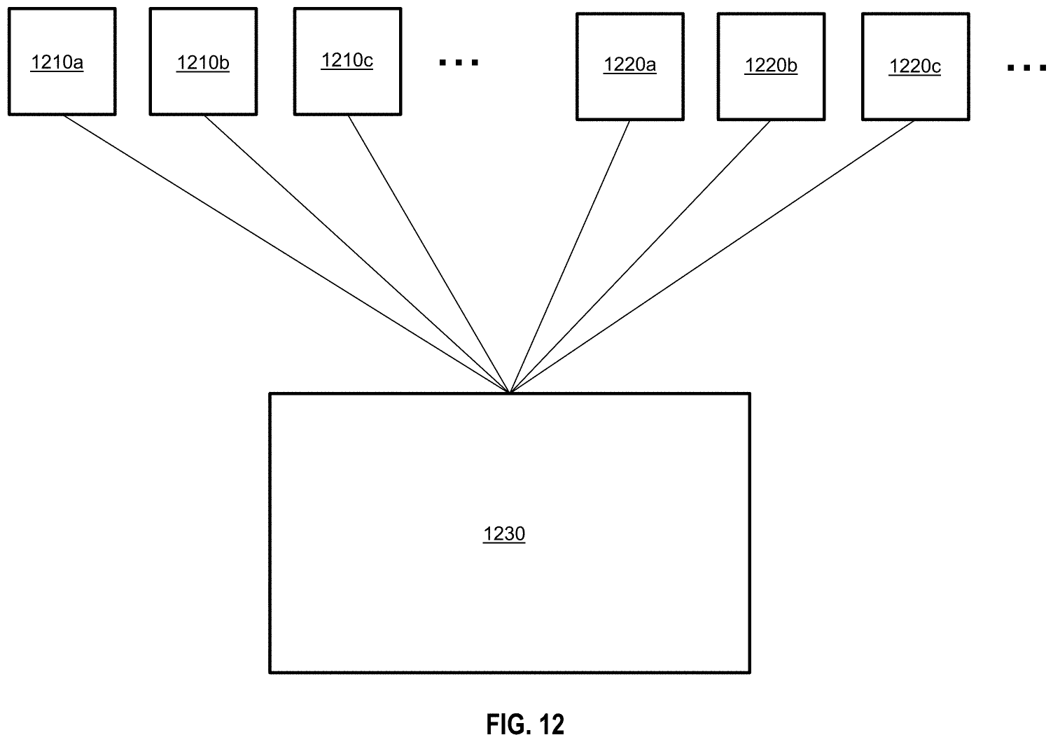

FIG. 12 shows an example of communication between different devices for a given entity, in accordance with embodiments of the invention. As previously described, one or more remote communication devices 1210a, 1210b, 1210c may communicate with one or more local communication devices 1220a, 1220b, 1220c. A remote communication device may be utilized by a remote individual. A local communication device may be utilized at a site of a medical procedure (e.g., operating room at a hospital), optionally by medical personnel.

An entity 1230 may facilitate the communications between the remote communication devices and the local communication devices. The entity may be a third party that is different from the remote individuals and/or health care facilities. The entity may run or operate a communication system. The communication facilitation entity may optionally provide a platform and/or infrastructure to facilitate the communications. In some instances, the communication facilitation entity may utilize cloud computing infrastructure to provide communication services. The entity may optionally provide software and/or applications that may facilitate the communications. The entity may provide a portal through which the local communication devices and the remote communication devices may connect. Tangible, non-transitory computer readable media may be provided comprising code, logic, or instructions that may facilitate the communications between the remote communication devices and the local communication devices.

Communications may be initiated by one or more local communication devices. For instance, the one or more local communication devices may initiate a call or communication to one or more remote communication devices. The remote communication devices may be able to accept, reject, and/or ignore the communication. Additionally and/or alternatively, communications may be initiated by one or more remote communication devices. For instance, the one or more remote communication devices may initiate a call or communication to one or more local communication devices. The local communication devices may be able to accept, reject and/or ignore the communication.

The entity may operate a video analysis system, as described herein. The entity may receive videos from one or more cameras at the various locations. The video analysis system may perform one or more video analysis steps as provided herein.

In some embodiments, a plurality of remote users may join a virtual session or workspace to collectively view one or more videos of a surgical procedure, and to collaborate with one another based on the one or more videos. The one or more videos may comprise a highlight video as described elsewhere herein. Such collaboration may involve, for example, a first remote specialist recording a portion of the one or more videos, telestrating on top of the recorded portion of the one or more videos, and streaming or broadcasting the recorded portion containing the one or more telestrations to a second remote specialist or at least one other individual. The at least one other individual may be, for example, someone who is either (a) remote from the healthcare facility in which the surgical procedure is being conducted, or (b) in or near the healthcare facility in which the surgical procedure is being conducted. As used herein, telestrating may refer to providing one or more annotations or markings to an image, a video, or a recording of a video that was previously streamed or that is currently being streamed live. As used herein, telestration may refer to one or more annotations or markings that can be provided or overlaid on an image, a video, or a recording of a video (e.g., using a finger, a stylus, a pen, a touchscreen, a computer display, or a tablet display). The telestrations may be provided based on a physical input, or based on an optical detection of one or more movements or gestures by the user providing the telestrations.

In some cases, while one or more videos of a live surgical procedure are being streamed, multiple specialists can join in on the virtual session to record various portions of the ongoing surgical procedure, telestrate on the recordings respectively captured by each specialist, and simultaneously stream back the recordings containing the telestrations to (i) the other specialists in the virtual session, or (ii) an individual who is in or near the healthcare facility in which the surgical procedure is being performed (e.g., the doctor or surgeon performing the surgical procedure). Such simultaneous streaming and sharing of the recordings containing the telestrations can allow the various remote specialists to compare and contrast their interpretations and evaluations of the surgical procedure, including whether or not a step is being performed correctly, and if the surgeon performing the procedure can make any adjustments or improvements to increase efficiency or minimize risk.

In some cases, the virtual session may permit the multiple specialists to simultaneously share their screens. In such instances, a first specialist can show a second specialist live telestrations that the first specialist is providing on the one or more videos while the second specialist also shows another specialist (e.g., the first specialist and/or another third specialist) telestrations that the second specialist is providing on the one or more videos. In some cases, the virtual session may permit the multiple specialists to simultaneously share individual recordings of the one or more videos. Such one or more individual recordings may correspond to different portions of the one or more videos, and may be of different lengths. Such individual recordings may be pulled from different cameras or imaging sensors used to capture the one or more videos of the surgical procedure. Such individual recordings may or may not comprises one or more telestrations, annotations, or markings provided by the specialist who initiated or captured the recording. For example, a first specialist may share a first recording corresponding to a first portion of the one or more videos, and a second specialist may share a second recording corresponding to a second portion of the one or more videos. The first portion and the second portion of the one or more videos may be selected by the specialist based on his or her interest or expertise in a particular stage or step of the surgical procedure. During such simultaneous sharing of individual recordings, a first specialist can show a second specialist live telestrations that the first specialist is providing on the one or more recorded videos while the second specialist also shows another specialist (e.g., the first specialist and/or another third specialist) telestrations that the second specialist is providing on the one or more recorded videos. Such simultaneous sharing of recordings and telestrations can allow the specialists to compare and contrast the benefits, advantages, and/or disadvantages of performing a surgical procedure in various different ways or fashions.

In some instances, simultaneous streaming and sharing of video recordings and live telestrations can allow a first remote specialist to see telestrations provided by a second and third remote specialist at the same time. In some cases, the second remote specialist can provide a first set of telestrations corresponding to a first method of performing a surgical procedure, and the third remote specialist can provide a second set of telestrations corresponding to a second method of performing the surgical procedure. The first remote specialist can view both the first and the second set of telestrations to compare the first and second methods of performing the surgical procedure. The first remote specialist can use both the first and the second set of telestrations to evaluate improvements that can be obtained (e.g., in terms of surgical outcome, patient safety, or operator efficiency) if the surgical procedure is performed in accordance with the various methods suggested or outlined by the telestrations provided by each remote specialist.

In some instances, simultaneous streaming and sharing of video recordings and live telestrations can allow a first user (e.g., a doctor or a surgeon performing a surgical procedure) to see telestrations provided by a second and third user at the same time. In some cases, the second user can provide a first set of telestrations corresponding to a first method of performing a surgical procedure, and the third user can provide a second set of telestrations corresponding to a second method of performing the surgical procedure. The first user can view both the first and the second set of telestrations to compare the first and second methods of performing the surgical procedure. The first user can use both the first and the second set of telestrations to evaluate improvements that can be obtained (e.g., in terms of surgical outcome, patient safety, or operator efficiency) if the surgical procedure is performed in accordance with the various methods suggested or outlined by the telestrations provided by each of the other users. The second user and the third user may be, for example, remote specialists who can provide feedback, commentary, guidance, or additional information to assist the first user while the first user is performing the surgical procedure, to provide additional training to the first user after the first user completes one or more steps of the surgical procedure, or to evaluate the first user's performance after completion of one or more steps of the surgical procedure.

In some instances, a first user (e.g., a first doctor or surgeon or medical specialist) can provide and share telestrations to show how a procedure should be completed. In some cases, a second user (e.g., a second doctor or surgeon or medical specialist) can provide separate telestrations (e.g., telestrations provided on a separate recording or a separate stream/broadcasting channel) to allow a third user (e.g., a third doctor or surgeon or medical specialist) to compare and contrast the various telestrations. In other cases, a second user (e.g., a second doctor or surgeon or medical specialist) can provide telestrations on top of the first user's telestrations to allow a third user (e.g., a third doctor or surgeon or medical specialist) to compare and contrast the various telestrations in a single recording, stream, or broadcast.

In some embodiments, the user or remote specialist who is sharing content (e.g., video recordings or telestrations) with the other users or specialists can share such content as a downloaded or downloadable file, or by providing access to such content via a server. Such server may be, for example, a cloud server.

In some cases, multiple users can telestrate the videos at the same time, and change the content of the videos by adding additional data or by changing some of the data associated with the videos (e.g., removing audio or post-processing the video). After the multiple users add additional data to the videos and/or change some of the data associated with the videos, the multiple users can re-broadcast the video containing the changed or modified content to other users (e.g., other remote specialists, or other individuals assisting with the surgical procedure). In some cases, the multiple users can provide further annotations or telestrations on top of the rebroadcasted videos containing various telestrations provided by other users, and to share such additional annotations or telestrations with the other users. In some cases, each of the users in the virtual session may provide their own telestrations in parallel and simultaneously share the telestrations such that each user sees multiple telestrations from other users corresponding to (i) the same portion or recording of a surgical video, (ii) various different portions or recordings of a surgical video or (iii) different views of the same portion or recording of a surgical video. Multiple users can telestrate at the same time and/or modify the telestrations that are provided by the various users at the same time. The telestrations may be provided on a live video stream of a surgical procedure or a recording (e.g., a video recording) of a surgical procedure. The multiple simultaneous telestrations by the multiple users may be provided with respect to the same live video stream or the same recording, in which case the multiple telestrations may be provided on top of one another. Alternatively, the multiple simultaneous telestrations by the multiple users may be provided with respect to different videos or recordings.

In some cases, the telestrations may be provided on a highlight video corresponding to various portions or sections of interest within a surgical video or a recording thereof. For example, a first user may provide a first set of telestrations associated with one or more portions or sections of interest within a surgical video. The telestrations may be shared, streamed, or broadcasted to other users. In some cases, multiple users may provide multiple sets of telestrations in parallel (e.g., separate telestrations on separate recordings, or a plurality of telestrations overlaid on top of each other). Such multiple sets of telestrations may be simultaneously streamed to and viewable by various users in the virtual session to compare and contrast various methods and guidance suggested or outlined by the various telestrations provided by the multiple users. In some cases, such multiple sets of telestrations may be simultaneously streamed to and viewable by various users in the virtual session to evaluate different ways to perform one or more steps of the surgical procedure to obtain different results (e.g., different surgical outcomes, or differences in operator efficiency or risk mitigation). In some cases, such multiple sets of telestrations may be simultaneously streamed to and viewable by various users in the virtual session so that the various users can see one or more improvements that can result from performing the surgical procedure in different ways according to the different telestrations provided by different users.

In some embodiments, the telestrations may be provided at a first time point of interest and a second time point of interest. The first time point of interest and/or the second time point of interest may correspond to one or more critical steps in the surgical procedure. The multiple users may provide multiple telestrations at the first time point of interest and/or the second time point of interest. The users may view the multiple telestrations simultaneously to see how outcomes or results at the second time point of interest change based on different actions taken at the first time point of interest. In some cases, the multiple telestrations may be provided with respect to different highlight videos so that a single user can see which steps or time points of a surgical procedure can impact a surgical outcome, and compare or contrast the various methods for performing such steps during such time points to improve the surgical outcome. As used herein, surgical outcome may correspond to an end result of a surgical procedure, a level of success of the surgical procedure, a level of risk associated with the performance of the surgical outcome, or an efficiency of the operator performing the surgical procedure.

In some embodiments, when a user (e.g., a specialist) telestrates on top of one or more videos or recordings, the user can share the one or more videos with other users (e.g., other specialists) at the same time. Further, the user may share multiple applications or windows at the same time along with the one or more videos or recordings having the telestrations provided by that user. This allows other users or specialists to view (i) the one or more videos or recordings having the telestrations and (ii) one or more applications or windows comprising additional information or content associated with the surgical procedure, in parallel or simultaneously. Such additional information or content may comprise, for example, medical or surgical data, reference materials pertaining to a performance of the surgical procedure or a usage of one or more tools, or additional annotations or telestrations provided on various videos or recordings of the surgical procedure. Allowing users or specialists to share one or more videos, applications, and/or windows at the same time with other users or specialists permits the other users or specialists to view, interpret, and analyze the shared videos or recordings containing one or more telestrations with reference to additional information or content. Such additional information or content can provide additional background or context for understanding, interpreting, and analyzing the shared videos or recordings and/or the telestrations provided on the shared videos or recordings.

Computer Control Systems

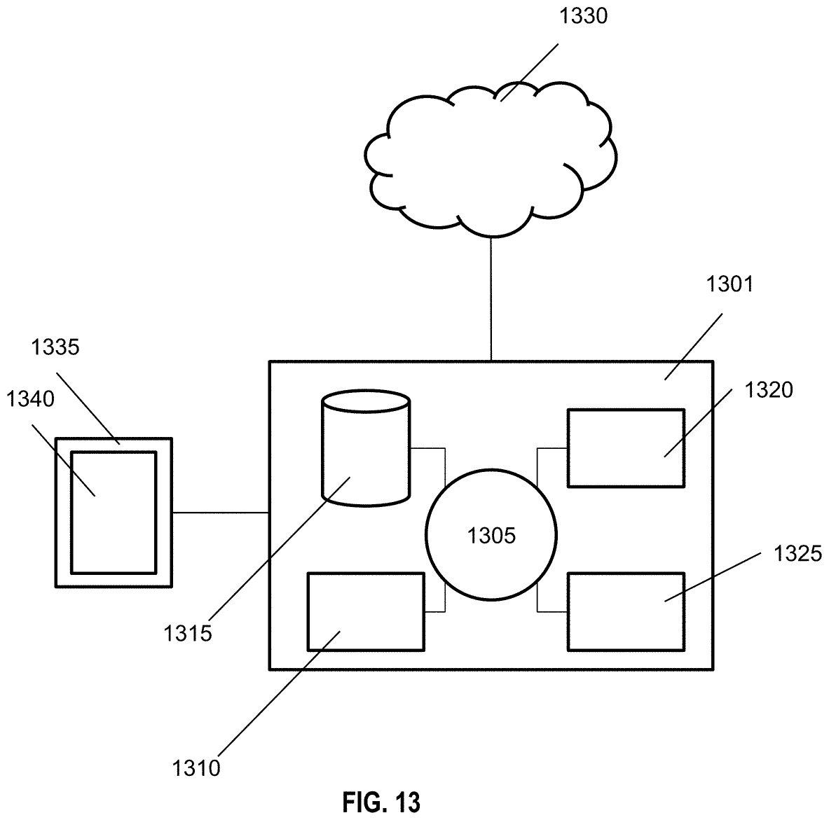

The present disclosure provides computer control systems that are programmed to implement methods of the disclosure. FIG. 13 shows a computer system 1301 that is programmed or otherwise configured to facilitate communications between vendor representatives and medical personnel that may need a vendor representative's support. The computer system may facilitate communications between a rep communication device and a local communication device. The computer system may automatically interface with one or more scheduling systems of one or more health care facilities. The computer system may access information about one or more vendors, such as one or more vendor representatives. The computer system may automatically determine one or more vendor representatives that may be suitable for providing support for a medical procedure utilizing a medical product. The computer system may optionally present one or more visual regions that allow the vendor representative to easily connect with the medical personnel. The computer system may comprise a rep communication device and/or a local communication device. The computer system may comprise, or be in communication with, one or more devices separate from the rep communication device and/or local communication device. The computer system can be an electronic device of a user or a computer system that is remotely located with respect to the electronic device. The electronic device can be a mobile electronic device.

The computer system 1301 may include a central processing unit (CPU, also “processor” and “computer processor” herein) 1305, which can be a single core or multi core processor, or a plurality of processors for parallel processing. The computer system also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet.

The network 1330 in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, capturing a configuration of one or more experimental environments; storing in a registry the experimental environments at each of one or more time points; performing one or more experimental executions which leverage experimental environments; providing outputs of experimental executions which leverage the environments; generating a plurality of linkages between the experimental environments and the experimental executions; and generating one or more execution states corresponding to the experimental environments at one or more time points. Such cloud computing may be provided by cloud computing platforms such as, for example, Amazon Web Services (AWS), Microsoft Azure, Google Cloud Platform, and IBM cloud. The network, in some cases with the aid of the computer system 1301, can implement a peer-to-peer network, which may enable devices coupled to the computer system to behave as a client or a server.

The CPU 1305 can execute a sequence of machine-readable instructions, which can be embodied in a program or software. The instructions may be stored in a memory location, such as the memory 1010. The instructions can be directed to the CPU, which can subsequently program or otherwise configure the CPU to implement methods of the present disclosure. Examples of operations performed by the CPU can include fetch, decode, execute, and writeback.

The CPU 1305 can be part of a circuit, such as an integrated circuit. One or more other components of the system can be included in the circuit. In some cases, the circuit is an application specific integrated circuit (ASIC).

The storage unit 1315 can store files, such as drivers, libraries and saved programs. The storage unit can store user data, e.g., user preferences and user programs. The computer system 1301 in some cases can include one or more additional data storage units that are external to the computer system, such as located on a remote server that is in communication with the computer system through an intranet or the Internet.

The computer system 1301 can communicate with one or more remote computer systems through the network 1330. For instance, the computer system can communicate with a remote computer system of a user (e.g., a user of an experimental environment). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1035 that comprises a user interface (UI) 1340 for providing, for example, selection of an environment, a component of an environment, or a time point of an environment. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1305. The algorithm can, for example, capture a configuration of one or more experimental environments; store in a registry the experimental environments at each of one or more time points; perform one or more experimental executions which leverage experimental environments; provide outputs of experimental executions which leverage the environments; generate a plurality of linkages between the experimental environments and the experimental executions; and generate one or more execution states corresponding to the experimental environments at one or more time points.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

We claim:

1. A method of creating a highlight video of a medical procedure, said method comprising:
recognizing one or more steps performed within a medical procedure based on video or audio data captured at the location of the medical procedure;
assessing an amount of time to perform each of the one or more steps performed within the medical procedure, and retrieving a predicted amount of time based at least in part on an anatomy type of a subject of the medical procedure;
comparing the amount of time to perform each of the one or more steps in relation to the predicted amount of time to perform each of the one or more steps; and
creating a highlight video of the medical procedure comprising video from the one or more steps where the amount of time to perform each of the one or more steps deviates from the predicted amount of time by more than a threshold.

2. The method of claim 1, further comprising creating model video with audio before the medical procedure of the predicted medical procedure state with spatial and temporal analysis to assist medical personnel with performance of the medical procedure.

3. The method of claim 1, further comprising creating automated documentation and transcripts using scene detection based on the video or audio data captured at the location of the medical procedure.

4. The method of claim 1, further comprising:
predicting subsequent steps during the medical procedure to provide guidance for the medical procedure;
indicating current and next steps during an active medical procedure; and
modifying subsequent steps based on steps performed during the medical procedure, based on a success or accuracy rate.

5. The method of claim 1, wherein the highlight video is created or displayed during the medical procedure.

6. The method of claim 1, wherein the highlight video is created or displayed after completion of the medical procedure.

7. The method of claim 1, wherein the highlight video comprises an analysis of how one or more of the steps in the highlight video may be improved, when the amount of time to perform each of the one or more steps exceeds the predicted amount of time by more than the threshold.

8. (canceled)

9. The method of claim 1, wherein the video data is captured with aid of a medical console capable of communicating with a remote device, wherein the medical console comprises at least one camera supported by a movable arm.

10. (canceled)

11. The method of claim 1, further comprising utilizing machine learning to recognize the one or more steps performed in the medical procedure and corresponding timing.

12. The method of claim 1, further comprising utilizing machine learning to determine the predicted amount of time to perform each of the one or more steps.

13. (canceled)

14. A system for creating a highlight video of a medical procedure, said system comprising one or more processors configured to, collectively or individually:
recognize one or more steps performed within a medical procedure based on video or audio data captured at the location of the medical procedure;
assess an amount of time to perform each of the one or more steps performed within the medical procedure;
compare the amount of time to perform each of the one or more steps in relation to a predicted amount of time to perform each of the one or more steps; and
create a highlight video of the medical procedure comprising video from the one or more steps where the amount of time to perform each of the one or more steps deviates from the predicted amount of time by more than a threshold.

15. The system of claim 14, further comprising one or more cameras or microphones configured to capture the video or audio data.

16. The system of claim 15, wherein the one or more cameras are supported by a medical console configured to communicate with a remote device.

17. (canceled)

18. (canceled)

19. (canceled)

20. (canceled)

21. (canceled)

22. A method for video collaboration, the method comprising:
(a) providing one or more videos of a surgical procedure to a plurality of users, wherein the one or more videos comprise at least one highlight video; and
(b) providing a virtual workspace for the plurality of users to collaborate based on the one or more videos, wherein the virtual workspace permits each of the plurality of users to (i) view the one or more videos or capture one or more recordings of the one or more videos, (ii) provide one or more telestrations to the one or videos or recordings, and (iii) distribute the one or more videos or recordings comprising the one or more telestrations to the plurality of users.

23. The method of claim 22, wherein the virtual workspace permits the plurality of users to simultaneously stream the one or more videos and distribute the one or more videos or recordings comprising the one or more telestrations to the plurality of users.

24. The method of claim 23, wherein the virtual workspace permits a first user to provide a first set of telestrations and a second user to provide a second set of telestrations simultaneously.

25. The method of claim 24, wherein the virtual workspace permits a third user to simultaneously view the first set of telestrations and the second set of telestrations to compare or contrast inputs or guidance provided by the first user and the second user.

26. (canceled)

27. (canceled)

28. (canceled)

29. The method of claim 24, wherein the first set of telestrations and the second set of telestrations are provided with respect to different videos or recordings captured by the first user and the second user.

30. The method of claim 24, wherein the first set of telestrations and the second set of telestrations are provided or overlaid on top of each other with respect to a same video or recording captured by either the first user or the second user.

31. (canceled)

32. The method of claim 22, wherein the virtual workspace permits the plurality of users to provide telestrations at the same time or modify the telestrations that are provided by one or more users of the plurality of users at the same time.

33. (canceled)
